(19) United States
(12) Patent Application Publication
(10) Pub. No.: US 2020/0315834 A1
(43) Pub. Date: Oct. 8, 2020

(54) External Nasal Dilator with Greater Breathable Surface Area

(71) Applicant: Joseph V. IERULLI, Bradenton, FL (US)

(72) Inventor: Joseph V. IERULLI, Bradenton, FL (US)

(73) Assignee: Corbett Lair Inc., Bradenton, FL (US)

(21) Appl. No.: 16/377,196

(22) Filed: Apr. 6, 2019

(51) Int. Cl. A61F 5/08

(52) U.S. Cl. CPC A61F 5/08

(57) ABSTRACT

An external nasal dilator comprising resilient and engagement elements is provided. A greater portion of the plan view surface area of the nasal dilator supports moisture vapor transmission (MVT) compared to prior art nasal dilators having a nearly identical plan view surface area and the same or similar functional resiliency. The engagement element surface area relative to the surface area of the resilient element of nasal dilators of the present invention is significantly greater compared to prior art nasal dilators having a nearly identical plan view surface area and the same or similar functional resiliency. In use the nasal dilator stabilizes and/or expands nasal passage outer wall tissues, and prevents said tissues from drawing inward during breathing.

### CONTINUITY AND CLAIM OF PRIORITY

This is an original U.S. patent application.

### FIELD

The present invention relates generally to medical devices, and more particularly to apparatus for and methods of supporting or dilating external tissue in humans. As disclosed and taught in the preferred embodiments, the tissue dilator devices are particularly suitable for, and are directed primarily to, external nasal dilators for supporting, stabilizing, and dilating nasal outer wall tissues adjacent and overlying nasal airway passages of the human nose. The United States Food and Drug Administration classifies the nasal dilator as a 510(K) Exempt [Medical] Device Class 1, product code LWF, regulation no. 874.3900.

### BACKGROUND

External nasal dilators (END) are well disclosed in the art and are widely available in retail consumer markets where they are generally referred to as nasal strips or nasal dilator strips. In use the END extends over the nose, flexed across the bridge and adhered to the skin surfaces on either side thereof.

The functional part of the END is at least one resilient member (synonymously referred to in the art as a spring, spring member, resilient band, resilient member band, spring band, or bridge) that extends along the length of the nasal dilator. When flexed, the resilient member exerts spring biasing forces that urge nasal passage outer wall tissues outward, stabilizing the outer walls and expanding, or dilating, the nasal passages underneath.

Stabilized nasal outer walls and/or dilated nasal passages increase nasal patency and reduce nasal airflow resistance, beneficially affecting nasal obstruction and nasal congestion primarily about the nasal valve and extending to the nostril opening. Stabilized nasal outer walls are less likely to collapse during inhalation. Dilated nasal passages have increased cross sectional area and greater nasal cavity volume. Stabilization and/or dilation, particularly at the nasal valve, results in a corresponding improvement in nasal airflow, which may have beneficial effects generally, may increase oxygen uptake, may improve sleep, may reduce sleep disturbances, or may improve nasal snoring or obstructive sleep apnea (OSA). External nasal dilators have been shown to have beneficial effects for athletes, particularly in sports where a mouth-guard is worn. For aerobic sports, nasal strips may delay onset of intra-oral breathing, and may create a subjective feeling of increased respiration, which may provide a psychological benefit in competition.

The most prevalent, common and widely available nasal dilators have two or three closely parallel resilient bands extending from end to end of the nasal dilator, and four corner tabs, x, as illustrated in FIGS. 18 and 19. They have historically accounted for the vast majority of all external nasal dilators sold in the U.S. consumer market since introduced to consumers in 1995, according to retail sales data as provided, for example, by the Nielsen Company, US, LLC retail measurement services. These nasal dilators are efficient in terms of size, shape, resiliency, efficacy and reliability. Their construction includes a relationship between the surface area that adheres the dilator to the nose and the surface area that acts to urge the nasal passage tissues outward.

Roughly half of all nasal strips currently sold consumers in the U.S. retail market are constructed using clear polyethylene (PE) film (also according to Nielsen data) to engage the nasal dilator to the nose; the other half use a nonwoven fabric. PE film is a moisture vapor barrier. That is, the film does not ‘breathe’, or allow moisture vapor—as from the skin of the nose of a user—to pass through its thickness. Additionally, the nasal dilator's plastic resilient member is also impermeable to moisture and air, so 100% of the PE nasal dilator surface area is non-breathable. Where nonwoven fabrics are used instead of PE, only about half of the surface area is breathable, primarily at corner tabs x, as seen in FIGS. 18 and 19.

Buildup of moisture vapor between the nasal dilator and the skin surface of the nose can cause itching and discomfort. Moisture vapor buildup may also cause adhesive failure and thus premature disengagement of the dilator from the skin surface of the nose. Premature disengagement is believed to be the most common complaint from nasal dilator users. However, for users with sensitive skin it is believed that this same buildup of moisture makes a PE film dilator easier to remove and less likely to irritate the skin upon removal. Irritation upon removal is believed to be the second most common user complaint.

Accordingly, there is an unmet need in the nasal strip market, particularly in view of the popularity of PE film nasal dilators, to provide breathable film-based nasal dilators, and to provide nasal dilators with greater breathable surface area while retaining the efficiency of current widely available nasal dilators.

Human skin surfaces have maximum breathability when not covered by an article, such as clothing or a medical device, or by a substance such as lotion or sunscreen. Adhesive articles made of breathable thermoplastic film are generally regarded as being more effective and comfortable on the skin than non-breathable film articles. Breathable films may include those that have an inherent Moisture Vapor Transmission Rate (MVTR), such as polyurethane (PU) film, or may include those that have an engineered MVTR, such as micro-porosity, made by perforating a film's thickness.

Several external nasal dilator prior art references teach that PU films are suitable nasal dilator material choices. However, nasal dilators constructed using PU film have not been heretofore available to consumers. Nasal dilators having a microporous PE film were briefly, but widely, available to U.S. consumers from about 1995 to about 1998.

The present invention provides new and nonobvious external nasal dilators that address unmet needs, that are commercially viable, and which may be mass produced on an economic scale.

### SUMMARY

Nasal dilators of the present invention have a significantly greater engagement element surface area relative to the resilient element surface area compared to prior art nasal dilators. That greater surface area may be made breathable so as to increase the nasal dilator's moisture vapor transmission rate (MVTR). Greater MVTR generally makes an article worn on the skin for any length of time more comfortable.

Nasal dilators of the present invention comprise an engagement element for securing the nasal dilator to the skin surfaces of a nose of a user, and a resilient element that provides resiliency, or spring biasing forces, for stabilizing or dilating the nasal passages. The engagement element defines a plan view surface area of the nasal dilator that is substantially the same as the most widely available prior art nasal dilators. The resilient element provides the same resiliency or spring biasing force as the prior art nasal dilators, but within a substantially lesser surface area.

Some embodiments include at least one of an opening formed in the engagement element whereby MVTR may be further improved.

### Terminology and Illustration Notes

The terms spring biasing, spring biasing force, spring force, resiliency, spring constant, etc. as used herein are generally synonymous. Nasal dilators of the present invention may generate spring biasing force in a range of from about 5 grams to about 60 grams. A preferred range is from about 15 grams to about 35 grams for non-athletes, and from about 25 grams to about 45 grams for use in training, conditioning and competition by athletes. Less than 15 grams of spring biasing may not provide enough stabilization or dilation for some users, while greater than 35 grams may be uncomfortable for non-athletic use, such as during sleep, work or study.

The nasal dilator resilient member is semi-rigid; it is flexible out-of-plane with very little or no in-plane elongation. Strictly speaking, the term resilient may be used to describe objects that exhibit either ‘flexure’ or ‘elasticity’. For purposes of the present invention, however, the terms resilient, resiliency, spring biasing, etc., mean flexure out-of-plane, in a direction perpendicular or oblique to the surface plane, while being substantially rigid in-plane. This is different from, for example, an elastic web that stretches in a direction parallel to its surface plane, even though both the elastic web and the nasal dilator resilient member may return at least substantially to their initial positions after stretch or flexure, respectively. Nasal dilators herein may be described as “capable of flexing” (when the dilator is in an initial or un-flexed position), or “flexed” (when the dilator is engaged to the nose of a user).

The present invention is not limited to the illustrated or described embodiments, which are examples of forms of the present invention. All structures and methods that embody similar functionality are intended to be covered hereby. The nasal dilators depicted, taught, enabled and disclosed herein represent new, useful and non-obvious nasal dilator devices having a variety of alternative embodiments. Some embodiments of the present invention may refer to, or cross reference, other embodiments. It may be apparent to one of ordinary skill in the art that nasal dilator features, construction or configuration may be applied, interchanged or combined between and among the preferred embodiments.

For descriptive clarity, certain terms may be used in the specification and claims: Vertical refers to a direction parallel to thickness, such as the thickness of a finished article, a member or component, or a laminate. Horizontal refers to length or longitudinal extent, such as that of a finished article or element thereof, or a direction parallel thereto. Lateral refers to width or lateral extent. Longitudinal also refers to length, perpendicular to width or lateral extent. A longitudinal centerline is consistent with the long axis of a finished device, element, member or layer, bisecting its width midway between the long edges. A lateral centerline bisects the long axis midway along its length, perpendicular to the longitudinal centerline. The terms upper and lower refer to object orientation, particularly in plan views, relative to the top and bottom of the drawing sheet.

Broken lines and dashed lines may be used in the drawings to aid in describing relationships or circumstances with regard to objects:
A broken line including a dash followed by three short spaces with two short dashes therebetween indicates separation for illustrative purposes, such as in an exploded view, or to indicate an object or objects removed or separated from one or more other objects, primarily for illustrative clarity.
A dashed line (sometimes referred to as a shadow line) of successive short dashes with short spaces therebetween may be used to illustrate an object or element generically, to illustrate one object underneath another, or to reference environment such as facial features; or for clarity, to show location, such as the space an object or structure will occupy, would occupy, did occupy or may occupy; or for illustrative purposes, to represent an object, structure, element or layer(s) as transparent so that other objects more pertinent to the discussion at hand may be highlighted or more clearly seen.
A broken line including a long dash followed by a short space, a short dash and another short space is used to call out a centerline or an angle, or to indicate alignment; when accompanied by a bracket, to call out a section, segment or portion of an object or a group of objects; to illustrate a spatial relationship between one or more objects or groups of objects, or to create separation between objects for the purpose of illustrative clarity.

In the drawings accompanying this disclosure like objects are generally referred to with common reference numerals or characters, except where variations of otherwise like objects must be distinguished from one another. Where there is a plurality of like objects in a single drawing figure corresponding to the same reference numeral or character, only a portion of said like objects may be identified. After initial description in the text, some reference characters may be placed in a subsequent drawing(s) in anticipation of a need to call repeated attention to the referenced object. Where a feature or element has been previously described, shadow lines (dashed lines) may be used to generically illustrate the feature or element. Drawings are rendered to scale, but may be enlarged from actual size for illustrative clarity. Thickness may be slightly exaggerated in perspective views for illustrative clarity.

### BRIEF DESCRIPTION OF DRAWINGS

FIG. 1 is a perspective view of a nasal dilator, in accordance with the present invention, seen in use on a nose of a user.

FIG. 2 is a three-quarter perspective view of the nasal dilator illustrated in FIG. 1.

FIG. 3 is a top plan view of the nasal dilator of FIG. 1, the drawing including dark parallel lines to illustrate defined regions of the nasal dilator.

FIG. 4 is a perspective view of a nasal dilator, in accordance with the present invention, seen in use on a nose of a user.

FIG. 5 is a top plan view of the nasal dilator of FIG. 4.

FIG. 6 is an exploded perspective view of a nasal dilator in accordance with the present invention.

FIG. 7 is an exploded perspective view of a nasal dilator in accordance with the present invention.

FIG. 8 is an exploded perspective view of a nasal dilator in accordance with the present invention.

FIG. 9 is a three-quarter perspective view of the nasal dilator of FIG. 5.

FIG. 10 is an exploded perspective view of a nasal dilator in accordance with the present invention.

FIG. 11 is an exploded perspective view of a nasal dilator in accordance with the present invention

FIG. 12 is a top plan view of a nasal dilator in accordance with the present invention.

FIG. 13 is a perspective view of the nasal dilator of FIG. 12, seen in use on a nose of a user.

FIG. 14 is a three-quarter perspective view of the nasal dilator of FIG. 12.

FIG. 15 is a perspective view of a nasal dilator, in accordance with the present invention, seen in use on a nose of a user.

FIG. 16 is a top plan view of the nasal dilator of FIG. 15.

FIG. 17 is a three-quarter perspective view of the nasal dilator of FIG. 15.

FIG. 18 is a top plan view of a three-band prior art nasal dilator.

FIG. 19 is a top plan view of a two-band prior art nasal dilator.

FIG. 20 is a top plan view of the periphery of the nasal dilators depicted in FIGS. 1-11 overlaid onto a top plan view of the periphery of the prior art nasal dilator depicted in FIG. 18.

FIG. 21 is a top plan view of the periphery of the nasal dilators depicted in FIGS. 12-17 overlaid onto a top plan view of the periphery of the prior art nasal dilator depicted in FIG. 19.

FIG. 22 shows fragmentary plan views of nasal dilators of the present invention aligned to fragmentary plan views of the prior art nasal dilators of FIGS. 18 and 19.

FIG. 23 is a fragmentary plan view of the nasal dilator of FIGS. 12-17, taken on an enlarged scale.

FIG. 24 is a fragmentary plan view of the nasal dilator of FIGS. 1-11, taken on an enlarged scale.

FIG. 25 is a fragmentary plan view of the prior art nasal dilator of FIG. 18, taken on an enlarged scale.

FIG. 26 is a fragmentary plan view of the nasal dilator of FIGS. 1-11, taken on an enlarged scale.

FIG. 27 is a fragmentary plan view of the prior art nasal dilator of FIG. 19, taken on an enlarged scale.

FIG. 28 is a fragmentary plan view of the nasal dilator of FIGS. 12-17, taken on an enlarged scale.

FIG. 29 is a top plan view of a nasal dilator in accordance with the present invention.

FIG. 30 is a top plan view of a nasal dilator in accordance with the present invention.

FIG. 31 is a fragmentary plan view of the nasal dilator of FIGS. 29-30, taken on an enlarged scale.

FIG. 32 is a three-quarter perspective view of the nasal dilator of FIG. 29.

FIG. 33 is a perspective view of the nasal dilator of FIG. 29, seen in use on a nose of a user.

FIG. 34 is a three-quarter perspective view of the nasal dilator of FIG. 30.

FIG. 35 is a perspective view of the nasal dilator of FIG. 30, seen in use on a nose of a user.

FIG. 36 is a top plan view of a nasal dilator in accordance with the present invention.

FIG. 37 is a top plan view of a nasal dilator in accordance with the present invention.

FIG. 38 is a fragmentary plan view of the nasal dilator of FIGS. 36-37, taken on an enlarged scale.

FIG. 39 is a three-quarter perspective view of the nasal dilator of FIG. 36.

FIG. 40 is a three-quarter perspective view of the nasal dilator of FIG. 37.

FIG. 41 is a perspective view of the nasal dilator of FIG. 37, seen in use on a nose of a user.

FIG. 42 is a perspective view of the nasal dilator of FIG. 36, seen in use on a nose of a user.

FIG. 43 is a top plan view of a nasal dilator in accordance with the present invention.

FIG. 44 is a three-quarter perspective view of the nasal dilator of FIG. 43.

FIG. 45 is a perspective view of the nasal dilator of FIG. 43, seen in use on a nose of a user.

FIG. 46 is a top plan view of a nasal dilator in accordance with the present invention.

FIG. 47 is a perspective view of the nasal dilator of FIG. 46, seen in use on a nose of a user.

FIG. 48 is a three-quarter perspective view of the nasal dilator of FIG. 46.

FIG. 49 is a top plan view of a nasal dilator in accordance with the present invention.

FIG. 50 is a top plan view of a nasal dilator in accordance with the present invention.

FIG. 51 is a three-quarter perspective view of the nasal dilator of FIG. 49.

FIG. 52 is a perspective view of the nasal dilator of FIG. 49, seen in use on a nose of a user.

FIG. 53 is a three-quarter perspective view of the nasal dilator of FIG. 50.

FIG. 54 is a perspective view of the nasal dilator of FIG. 50, seen in use on a nose of a user.

FIG. 55 is a top plan view of a nasal dilator in accordance with the present invention.

FIG. 56 is a top plan view of a nasal dilator in accordance with the present invention.

FIG. 57 is a perspective view of the nasal dilator of FIG. 55, seen in use on a nose of a user.

FIG. 58 is a three-quarter perspective view of the nasal dilator of FIG. 55.

FIG. 59 is a perspective view of the nasal dilator of FIG. 56, seen in use on a nose of a user.

FIG. 60 is a three-quarter perspective view of the nasal dilator of FIG. 56.

FIG. 61 is a top plan view of a nasal dilator in accordance with the present invention.

FIG. 62 is a perspective view of the nasal dilator of FIG. 61, seen in use on a nose of a user.

FIG. 63 is a three-quarter perspective view of the nasal dilator of FIG. 61.

FIG. 64 is a top plan view of a nasal dilator in accordance with the present invention.

FIG. 65 is a perspective view of the nasal dilator of FIG. 64, seen in use on a nose of a user.

FIG. 66 is a three-quarter perspective view of the nasal dilator of FIG. 64.

### DETAILED DESCRIPTION

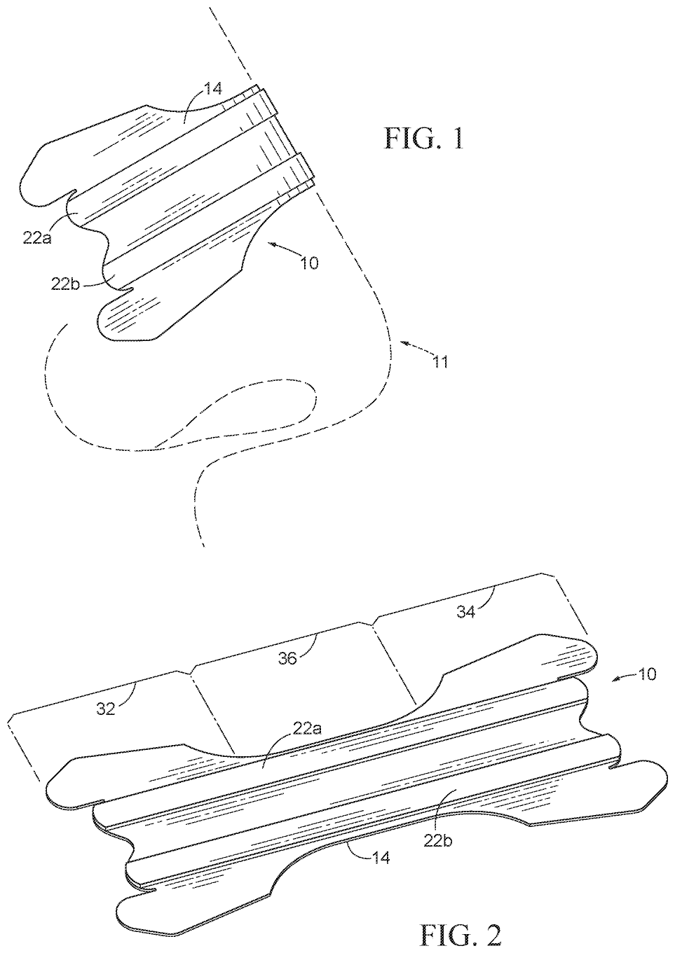

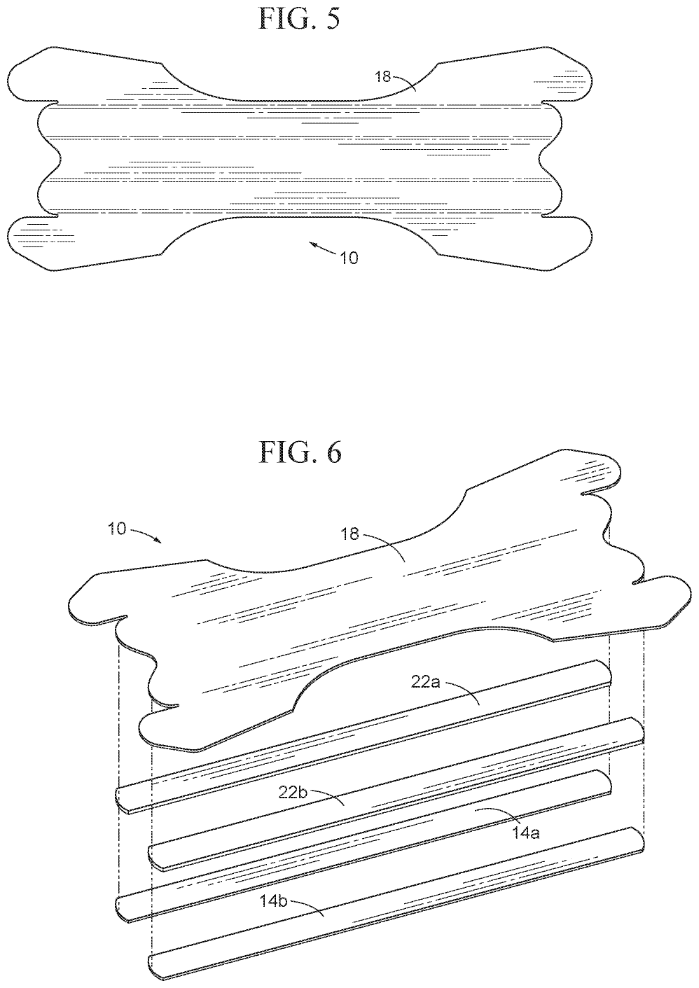

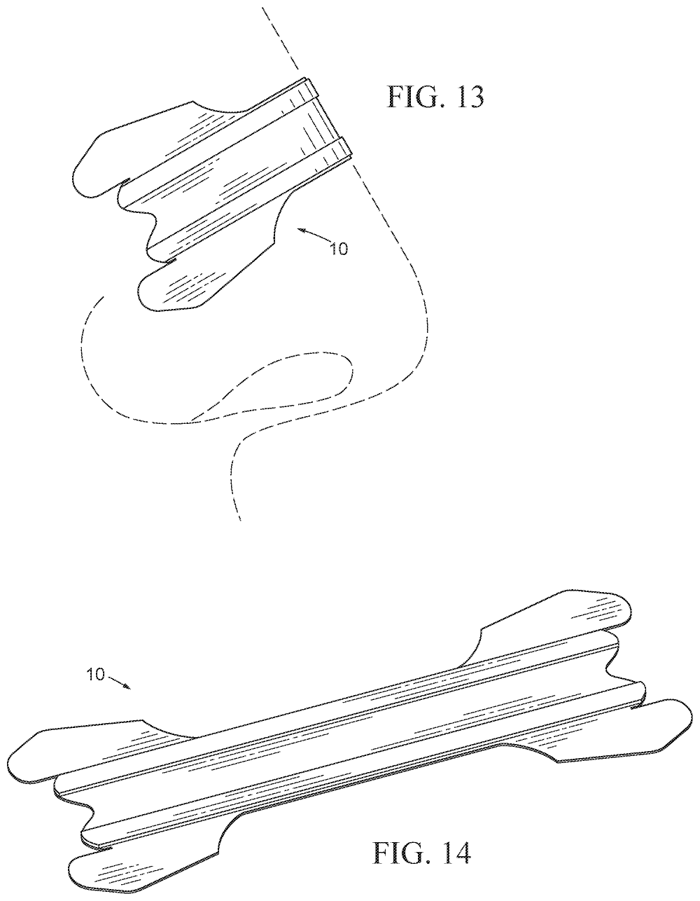

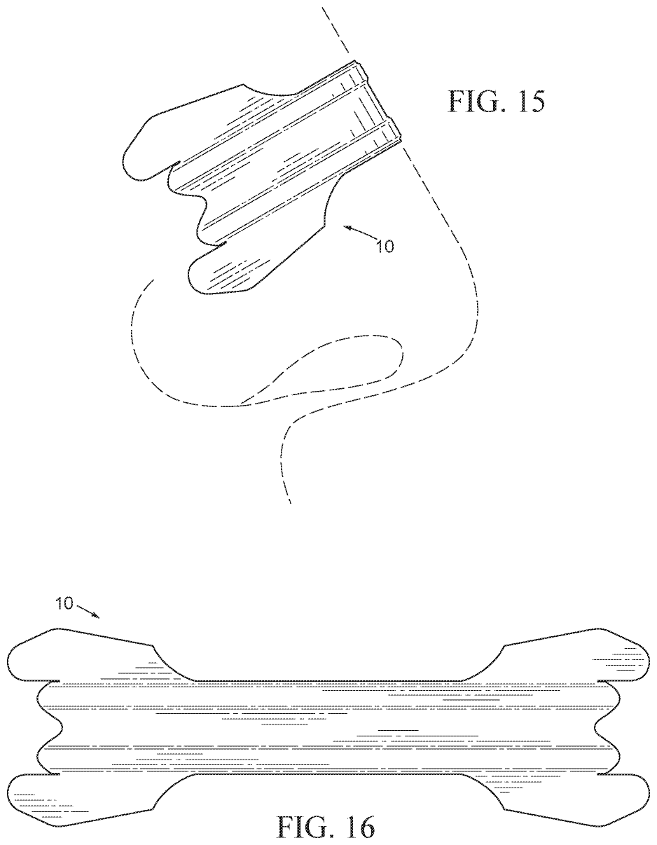

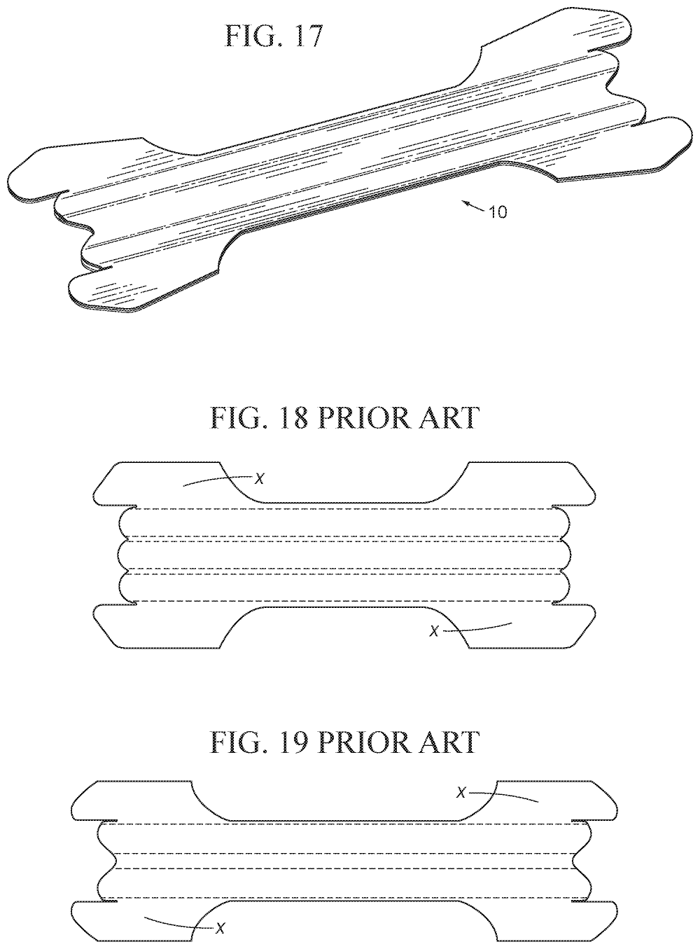

As particularly seen in FIGS. 1-17 a nasal dilator, 10, comprises a laminate of vertically stacked material layers that may include: a base layer comprising at least one base member, 14 (if a plurality, 14a, 14b, etc.); a resilient layer most preferably comprising two resilient members 22a and 22b; a cover layer comprising at least one cover member, 18. A protective layer of release liner, not shown, removably covers exposed adhesive from dilator 10 preliminary to application to nose 11.

Dilator layers may be secured to each other by any suitable means such as stitching or fastening, heat or pressure bonding, ultrasonic welding, or the like, but are preferably laminated by an adhesive substance disposed on at least one flat surface side of at least one layer.

The plan view periphery and total surface area of dilator 10 may be defined by either the base layer or cover layer. The base and cover layers, either individually or combined, together with a biocompatible adhesive disposed thereon for engaging the skin, provide the engagement element of dilator 10.

As seen in FIG. 2, the vertically stacked layers of dilator 10 form a unitary, or single body, truss. Brackets and broken lines delineate approximate boundaries between regions that extend laterally across the width of dilator 10. These regions include a first end region, 32, a second end region, 34, and an intermediate region, 36.

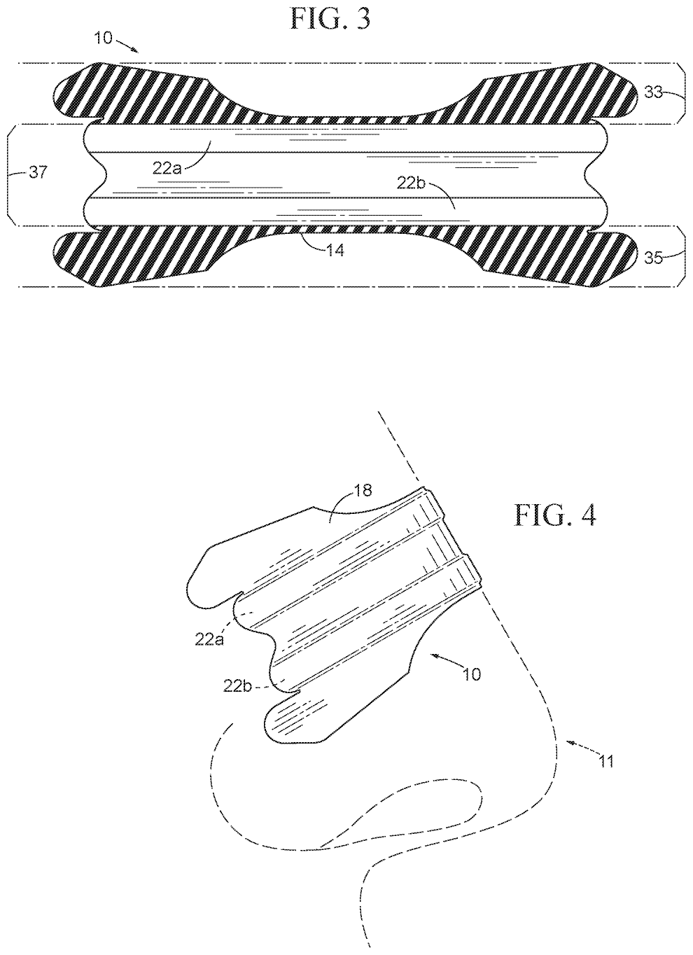

In FIG. 3, brackets and broken lines delineate specific boundaries between regions that extend fully along the length of dilator 10, including a first corner tab region, 33, a second corner tab region, 35, and a body region, 37. For illustrative emphasis the surface areas of corner tab regions 33 and 35 are marked by parallel thick dark lines. Corner tab regions 33 and 35 are positioned immediately adjacent each long edge of body region 37, abutting thereto. Resilient members 22a and 22b extend along body region 37, positioned in between its long edges immediately adjacent to the inside of each long edge.

The preferred materials for the base and cover members may be selected from a range of widely available, preferably medical grade, flexible fabrics or thermoplastic films that are breathable or permeable. The most preferable fabric is a synthetic nonwoven, and the most preferred thermoplastic film is from the class of polyurethane (PU) films from about 20 microns to about 60 microns thick. However, a PE film treated with a plurality of microporous openings may also be used.

An MVTR of at least 200 grams per square meter per 24 hours (GSM/24 hr.), is a most preferred minimum for the base and cover member materials. MVTR may be determined by a standard test such as, for example, ASTM F1249, ASTM E96, or ASTM2622. A layer of pressure sensitive adhesive, preferably biocompatible with external human tissue, may be disposed on a flat surface side of the preferred material and covered by a protective, removable, release liner. The disposition of adhesive may be fully coextensive with the preferred material surface area. To increase MVTR, the disposition may be pattern-coated such that a lesser amount of adhesive covers the entire surface area.

The preferred material for resilient members 22 is a thermoplastic resin that may be selected from a range having flexural, tensile and elastic moduli so as to have substantial in-plane rigidity and out-of-plane flexibility at a thickness preferably greater than 0.010″. A most preferred thermoplastic material is a biaxially oriented polyester resin, Poly(ethylene terephthalate) or PET (or boPET). PET is used in a number of medical device applications, is particularly suitable because of its resiliency or spring biasing properties, and is widely available as a medical/industrial commodity.

It should be briefly noted that it may be possible to utilize laser technology to perforate otherwise impermeable thermoplastic resin materials. However, the process may be unsuitable for nasal dilator manufacture on the economic scale required for wide commercial distribution. Furthermore, the perforations could reduce resiliency, requiring that the resilient member have a greater surface area to make up for the deficit.

The vertically stacked layers of dilator 10 may be arranged in several ways. The stacking order of dilator layers, including a peripheral defining layer, may be determined by the preferred material used.

For example, in FIGS. 1-3 base member 14 is the peripheral defining layer and is preferably formed from a thermoplastic film, most preferably a PU film. A cover layer is eliminated, so resilient members 22a and 22b are uppermost in the stacking order of layers and are exposed and visible. In FIGS. 4-7, for example, cover member 18 is the peripheral defining layer. It may be formed from either thermoplastic film or a nonwoven fabric and is uppermost in the stacking order. Where cover member 18 is the peripheral defining layer, base member 14 is most preferably formed from a nonwoven fabric.

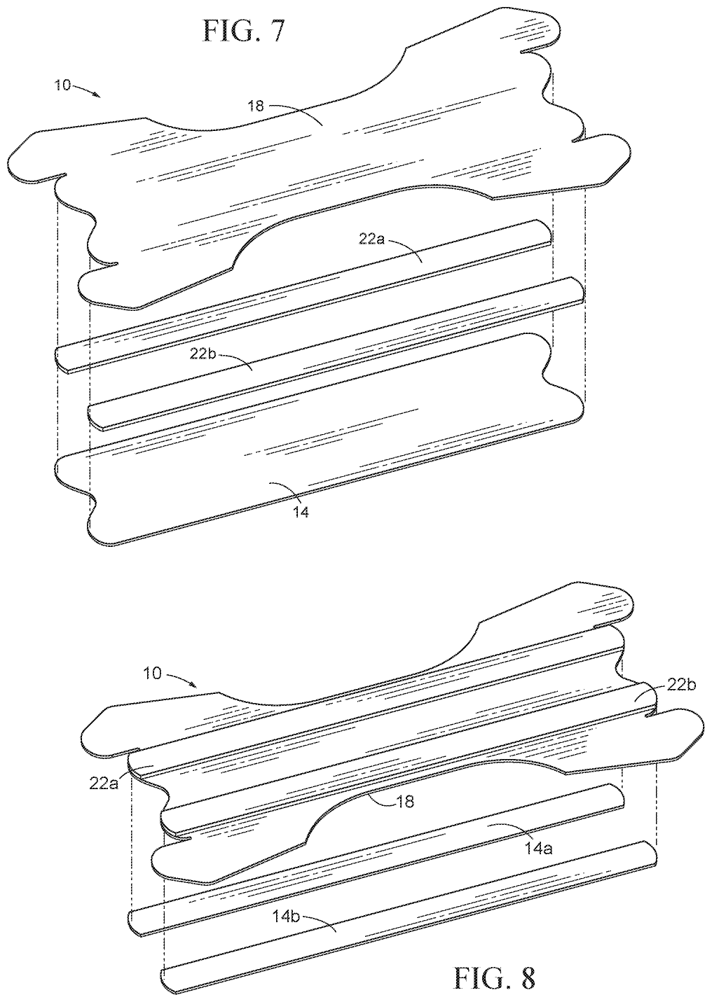

As seen in FIG. 6, the base and resilient layers may have identical plan view peripheries. That is, base members 14a and 14b are coextensive with the surface areas of resilient members 22a and 22b, respectively. Alternatively, base members 14a and 14b may be each slightly wider than either resilient member (not shown). Further alternatively, a single base member 14 may have a periphery encompassing both flat surface areas of resilient members 22, as seen, for example, in FIG. 7.

Where the base layer is made of a flexible fabric and has significantly less surface area than the cover layer, adhesive on the skin-engaging side of the base layer may be optionally eliminated. With or without adhesive, the base layer may also serve as a compressible buffer between the nasal dilator and the skin engaged thereby.

Yet another stacking order is illustrated in FIG. 8. Cover member 18 is preferably formed from a PU film; base members 14a and 14b are secured to the underside of cover member 18, and resilient members 22a and 22b are secured on top of cover member 18. Base members 14a and 14b are aligned with the resilient members, and are intended to absorb moisture vapor from the skin, which will not pass through the impermeable resilient members. Base members 14a and 14b are most preferably formed from a flexible spun-laced nonwoven fabric.

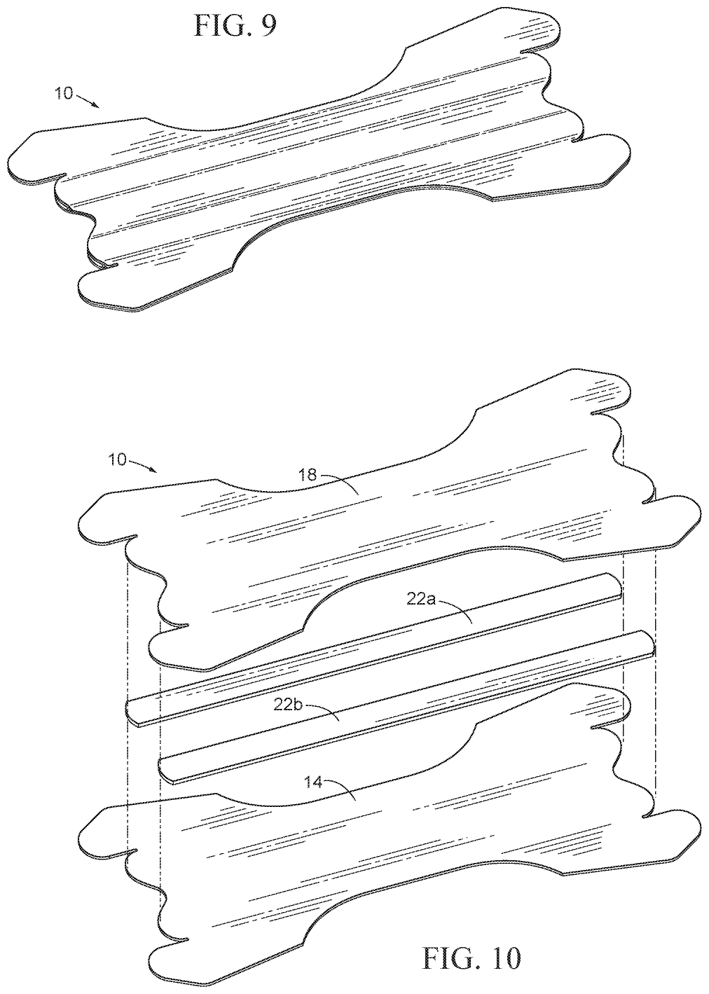

Alternatively, base member 14 and cover member 18 may have substantially similar or identical peripheries, as seen, for example, in FIGS. 9-10. In this instance, either base member 14 or cover member 18 may be formed from either thermoplastic film or nonwoven fabric. However, a single peripheral defining layer is most preferred.

Where it is formed from a PU film, the layer so formed most preferably has a sufficient thickness so as to be dimensionally stable without use of a supplemental carrier liner or stabilizing material layer. It may be apparent to one of ordinary skill in the art that unsupported ultra-thin PU film, for example, typically having a thickness of about 15 microns, is generally difficult to handle, prone to curling in on itself, and thus must be supported by a supplemental carrier liner until the article in question is secured to the skin. Accordingly, a single peripheral defining layer formed from ultra-thin PU film may be less preferable.

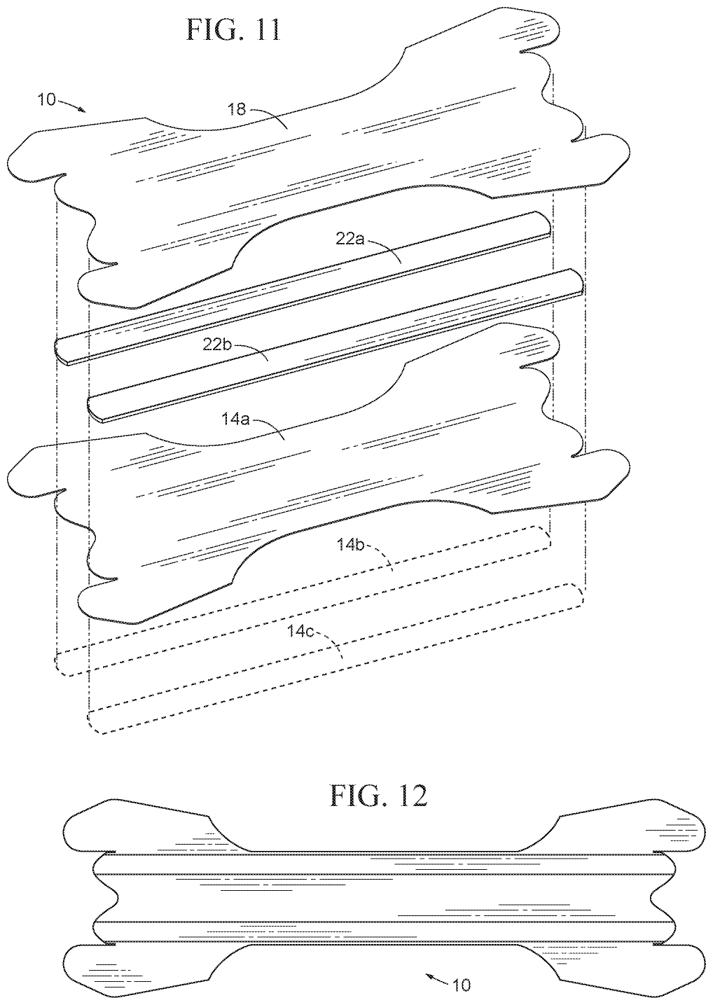

Nonetheless, FIG. 11 illustrates that resilient members 22a and 22b may be sandwiched between cover layer 18 and base layer 14 each formed from thin or ultra-thin PU film (from about 12 to about 19 microns thick for each of the two layers, for example), such that the combined thickness thereof renders dilator 10 dimensionally stable when the protective layer of release liner is removed from dilator 10 preliminary to application to nose 11. Base member 14a preferably has the same or similar periphery as cover member 18, and may have a somewhat lesser surface area. FIG. 11 further illustrates that base members 14b and 14c, depicted in shadow lines, may be optionally placed on the underside of base member 14a, aligned to the surface areas of resilient members 22a and 22b, so as to absorb moisture vapor from the skin as described hereinbefore.

The range of spring biasing force provided by resilient members 22a and 22b combined is most preferably consistent with that of the prior art nasal dilators shown in FIGS. 18 and 19, which have three or two resilient member bands, respectively. U.S. Pub. No. 20110000483 teaches that “In one embodiment, the total spring force delivered by the resilient element as a whole should be from 25 gm to about 35 gm.” U.S. Pat. No. 5,533,503 teaches that “The nasal dilator 10, of the present invention, is constructed to produce from 20 to 30 grams of dilating spring biasing force . . . .”

Accordingly, dilator 10 depicted in FIGS. 1-11 most preferably provides spring biasing force in a range of from about 25 grams to about 35 grams, consistent with the 3-band prior art nasal dilator shown in FIG. 18. Dilator 10 depicted in FIGS. 12-17 most preferably provides spring biasing force in a range of from about 20 grams to about 30 grams, consistent with the 2-band prior art nasal dilator shown in FIG. 19.

Spring biasing force is determined by resilient member dimensions. For example, one to three resilient members formed from PET, each measuring about 2.0″ in length, having a width/combined width of about 0.25″, and a thickness of 0.010″, generate roughly 25 grams of spring biasing force. Resilient members 22a and 22b each preferably have a width less than about 0.12″, and may have a thickness greater than 0.010″ so as to arrive at the preferred range of spring biasing force. If necessary, two or more resilient members may be overlaid or stacked one atop another so as to arrive at a combined thickness greater than 0.010″. Where resilient members 22 are stacked or overlaid, their length and width are preferably substantially the same.

As noted hereinbefore, nasal dilators of the present invention are drawn to scale. The prior art nasal dilators shown in FIGS. 18 and 19, being widely available to the public and so physically measurable, are also drawn to scale. The prior art nasal dilators and nasal dilators of the present invention are drawn to size relative to each other, particularly as seen in those drawing figures in which they are specifically compared, namely FIGS. 18-22, 25-26, and 27-28.

In that regard, FIG. 20 shows a periphery, p, of dilator 10 as shown in FIGS. 1-11, overlaid onto the periphery of the prior art 3-band nasal dilator, which is depicted in dashed lines. Similarly, FIG. 21 shows periphery p of dilator 10 as shown in FIGS. 12-17, overlaid onto the periphery of the prior art 2-band nasal dilator, also depicted in dashed lines. It should be visually apparent that the overall length and width of the nasal dilators and their peripheries are substantially the same. Furthermore, small differences between the dilator peripheries substantially offset, such that the surface areas of the prior art nasal dilators and nasal dilator 10 are nearly identical.

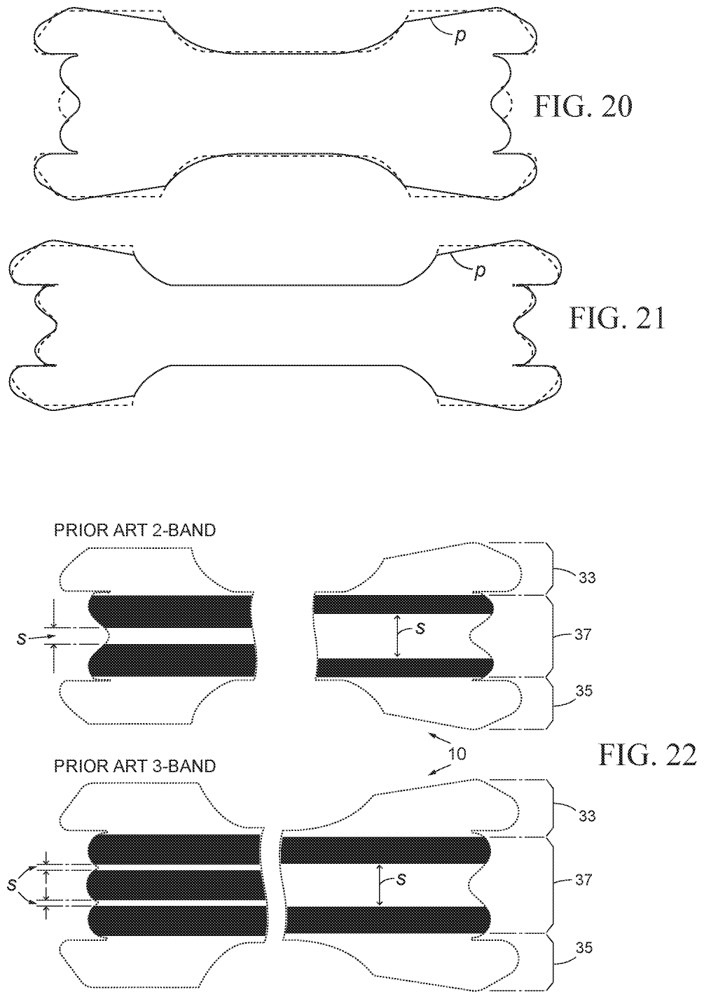

It may also be visually apparent that the width of body region 37 and corner tab regions 33 and 35 of dilator 10 are substantially the same as corresponding regions of the prior art nasal dilators. In this regard FIG. 22 shows fragmentary plan views of dilator 10 aligned to corresponding fragmentary plan views of the prior art nasal dilators. For all dilators, the width of body region 37 is about 45% of the dilator overall width; corner tab regions 33 and 35, combined, are about 55% of dilator overall width.

Thus far it has been demonstrated that resiliency (spring biasing force), periphery, overall surface area, body region width and corner tab region width are substantially the same, if not nearly identical, between nasal dilators of the present invention and the prior art nasal dilators.

FIG. 22 shows that resilient members 22 of dilator 10 occupy significantly less surface area than resilient members of the prior art nasal dilators, which is discussed further hereinbelow. (Resilient members are depicted in solid black for illustrative emphasis.) Additionally, a lateral distance, s, extends between the inside long edges of the resilient members. Distance s is about 2.7 times greater in dilator 10 compared to the 2-band prior art nasal dilator, and about 3.6 times greater than the prior art 3-band nasal dilator. The relative narrow width of the resilient members and the spacing between them denoted by distance s creates a substantial surface area within body region 37 that may be permeable rather than impermeable.

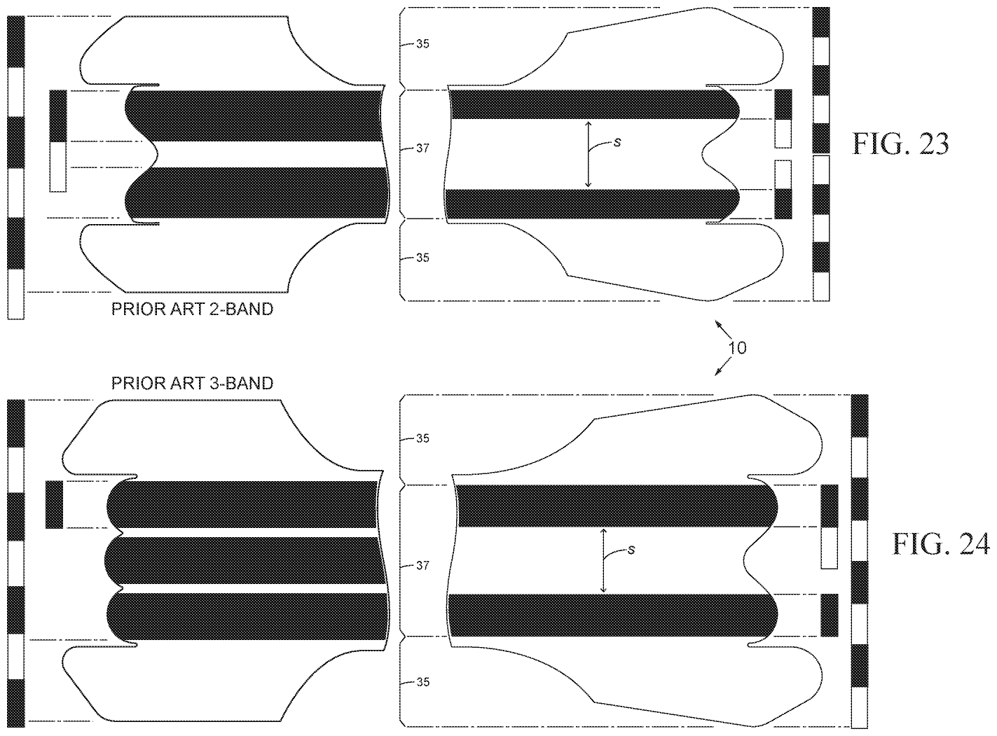

Since resilient members of both the prior art nasal dilators and dilator 10 extend fully from end to end, width comparisons may be viewed as one measure of the difference between impermeable (resilient member) surface area and permeable (engagement element) surface area, the latter being greater in dilators of the present invention compared to the prior art nasal dilators. To further compare widths, FIGS. 23 and 24 show a fixed unit of measure represented by black and white blocks. Each block unit is equal to the width of one resilient member of the nasal dilator adjacent thereto.

FIG. 23 shows distance s of dilator 10 being greater than two block units. For the prior art 2-band nasal dilator, space between the two resilient members is less than one-half unit. For dilator 10, one resilient member width is equal to about one-tenth (9.9%) of the overall width of dilator 10; both resilient members combined are about one-fifth (19.8%) thereof. The corresponding prior art 2-band nasal dilator resilient member width is greater, about two-elevenths (18.3%) of overall dilator width; both resilient members combined are slightly more than two-fifths (36.6%) thereof. The prior art nasal dilator resilient members' combined width occupies 1.85 times more of the dilator's overall width than do resilient members of the present invention. (Conversely, the combined width of resilient members of the present invention occupy 1.85 times less of the dilator's overall width than the resilient members of the prior art nasal dilator.)

Similarly, in FIG. 24, distances of dilator 10 is about one and one-half block units. For the prior art 3-band nasal dilator, combined space between the resilient members is less than one-half unit. For dilator 10, one resilient member width is equal to about one-eighth (12.6%) of the overall width of dilator 10; both resilient members combined are about one-fourth (25.1%) thereof. The corresponding prior art 3-band nasal dilator resilient member width is about one-seventh (14.5%) of overall dilator width; both resilient members combined are slightly less than three-sevenths (43.6%) thereof. The individual resilient member widths for both dilators are similar, but dilator 10 uses two resilient members instead of three. So, the three prior art resilient members' combined width occupies about 1.74 times more of the dilator's overall width. (Conversely, the combined width of two resilient members of dilator 10 occupy 1.74 times less of the dilator's overall width than do three resilient members of the prior art nasal dilator.)

Comparing body region width to combined resilient member width, FIG. 23 shows that resilient members 22a and 22b occupy about 45.2% of the body region width. Thus, body region width is about 2.2 times greater than the combined resilient member width. However, for the adjacent prior art 2-band nasal dilator, body region width is just 1.26 times the combined width of both resilient members; both resilient members combined are about 79.4% of body region width. The prior art resilient members thus occupy about 1.75 times more of the body region width than do resilient members of corresponding dilator 10 adjacent thereto.

Similarly, FIG. 24 shows that resilient members 22a and 22b occupy about 55.2% of body region width. Thus, body region width is about 1.8 times greater than the combined resilient member width. However, for the adjacent prior art 3-band nasal dilator, body region width is just 1.13 times the combined width of three resilient members; the three resilient members combined are about 88.2% of body region width. The prior art resilient members combined thus occupy about 1.6 times more of body region width than do the two resilient members of corresponding dilator 10 adjacent thereto.

Table 1.0 summarizes the aforementioned width comparisons:

TABLE 1.0

| | dilator 10 | prior art 2- | dilator 10 | prior art 3- |
|---|---|---|---|---|
| | FIG. 23 | band, FIG. 23 | FIG. 24 | band, FIG. 24 |
| distance s | >2 units | <1/2 unit | ~1.5 units | <1/2 unit |
| one resilient member | 1/10th | 2/11th | ⅛th | 1/7th |
| width to dilator width | (9.9%) | (18.3%) | (12.6%) | (14.5%) |
| combined resilient | ⅕th | 4/11th | ¼th | 3/7th |
| member widths to | (19.8%) | (36.6%) | (25.1%) | (43.6%) |
| dilator width | 1.85 × less | 1.85 × more | 1.74 × less | 1.74 × more |
| body region width > | 2.2 × | 1.26 × | 1.8 × | 1.13 × |
| combined resilient | greater | greater | greater | greater |
| member widths | | | | |
| combined resilient | 45.2% | 79.4% | 55.2% | 88.2% |
| member widths as a % | 1.76 × less | 1.76 × more | 1.6 × less | 1.6 × more |
| of body region width | | | | |

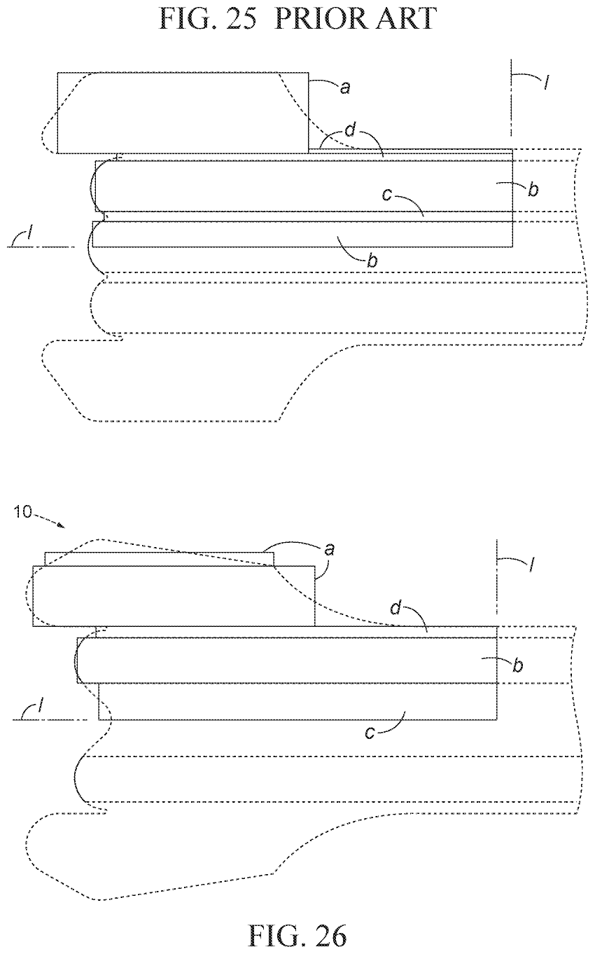

Further comparison of differences in impermeable resilient member surface area and permeable engagement element surface area is shown in FIGS. 25-28. To help visualize surface area comparison, various-sized rectangles are overlaid onto one quadrant of each nasal dilator whereby to measure particular surface areas: corner tab, a; resilient member, b; space between resilient members, c; and a narrow space, d, between corner tab a and resilient member b. Since the nasal dilators are symmetric about their longitudinal and lateral centerlines, 1, and all four quadrants are mirror images, only one quadrant is measured. According to these measurements, the total plan view surface area of dilator 10 (as seen in FIGS. 1-11) is about 2.7% less than the 3-band prior art nasal dilator (seen in FIG. 18), while the total plan view surface area of the 2-band prior art nasal dilator is about 0.5% less than dilator 10 (as shown in FIGS. 12-17).

Surface areas a, c and d are part of the engagement element extending in between and outward from resilient member surface areas b. As discussed hereinbefore, surface areas a, c and d are permeable, allowing moisture vapor to pass from the skin therethrough (i.e., MVTR); surface areas b do not allow moisture vapor transmission, regardless of the permeability of the engagement material to which the resilient member is secured. Note that surface areas a and d correspond to corner tab regions, which, combined, average about 40.1% of the total plan view surface area for both the prior art nasal dilators and dilator 10. Surface area c corresponds to the body region, which averages 59.1% thereof.

Overall, dilator 10 has roughly 1.4 times more permeable engagement element surface area, and from about 1.6 to 1.7 times less impermeable surface area, compared to the prior art nasal dilators. However, nearly all of the greater permeable surface area is within body region 37. There dilator 10 has from about 2.8 to about 3.6 times more permeable surface area. The ratio of permeable to impermeable surface area for the prior art nasal dilators is roughly 1:1, while for dilator 10 it's from about 2.1:1 to about 2.5:1, which is about 2.3 to 2.4 times greater. Specific detail follows, summarized in Tables 2.0-2.3.

Beginning with the prior art 3-band nasal dilator, FIG. 25 shows engagement element surface area c being about 7% of total plan view surface area. Surface areas a and d combined are about 40.1% thereof. Added together, surface areas a, c and d are about 47% of the total plan view surface area. Resilient member surface area b is about 53% of the total plan view surface area. So, the ratio of permeable engagement element surface area to non-permeable resilient member surface area is not quite equal, at about 0.9:1 (47.1/52.9=0.890 to be more exact).

FIG. 26 shows corresponding dilator 10 (as illustrated in FIGS. 1-11). Engagement element surface area cis almost 3.6 times greater than the prior art 3-band nasal dilator, and is about 25% of total plan view surface area. Surface areas a and d combined are about 42.4% of the total plan view surface area. Added together, areas a, c and d are about 67.4% of total plan view surface area. Resilient member surface area b is about 32.6% of the total plan view surface area, which is 1.62 times less than the prior art 3-band nasal dilator. The ratio of permeable engagement element surface area to non-permeable resilient member surface area is just over 2:1 (67.4/32.6=2.068 to be more exact). That ratio is more than double—about 2.3 times greater—than the 0.9:1 ratio of the prior art 3-band dilator.

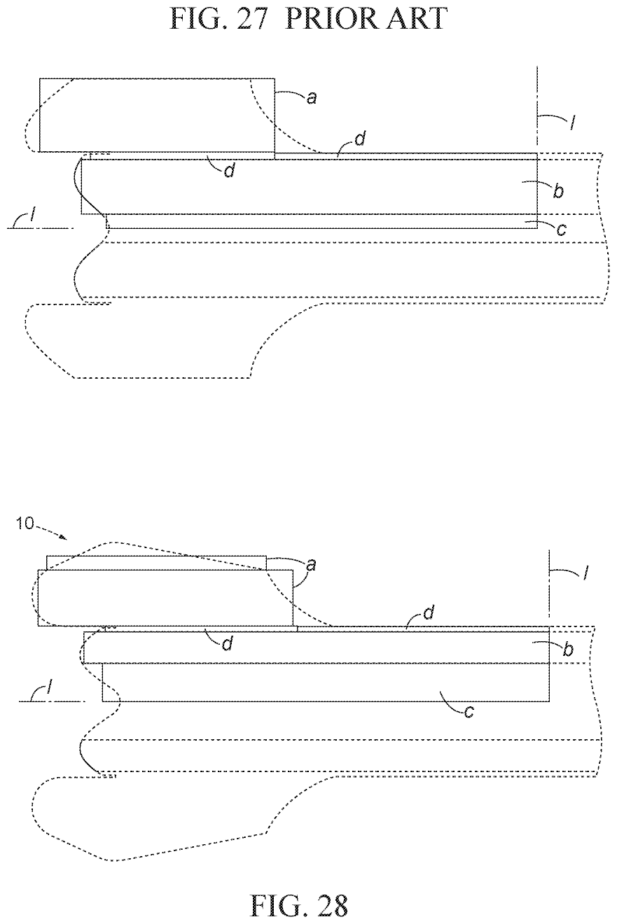

Turning now to the prior art 2-band nasal dilator, FIG. 27 shows engagement element surface area c being about 12% of total plan view surface area. Surface areas a and d combined are about 39.4% thereof. Added together, surface areas a, c and d are about 51.4% of total plan view surface area. Resilient member surface area b is about 48.6% of the total plan view surface area. So, the ratio of permeable engagement element surface area to non-permeable resilient member surface area is about equal at 1:1 (51.4/48.6=1.06 to be more exact).

FIG. 28 shows corresponding dilator 10 (as seen in FIGS. 12-17). Engagement element surface area c is about 2.8 times greater than the corresponding prior art 2-band nasal dilator, and is about 33% of total plan view surface area. Surface areas a and d combined are about 38.5% thereof. Added together, surface areas a, c and d are about 71.6% of total plan view surface area. Resilient member surface area b is about 28.4% of the total plan view surface area, which is about 1.7 times less than the corresponding prior art 2-band nasal dilator. So, the ratio of permeable engagement element surface area to non-permeable resilient member surface area is about 2.5:1 (71.6/28.4=2.52 to be more exact). That ratio is nearly 2.4 times greater than the 1:1 ratio of the prior art 2-band dilator.

Table 2.0 summarizes the aforementioned a-d surface areas. Additionally, resilient member surface area b and engagement element surface area c combined reflect the total surface area for body region 37 (shown in the last column). Engagement element surface areas a and d combined reflect the surface area for corner tab region 33 or 35 (shown in the first column). Adding the first and last columns together equals 100% of the total nasal dilator plan view surface area. Extrapolating from Table 2.0 shows that permeable surface area makes up from about 43% to 54% of body region 37 (25.0/57.6=43.4 and 33.1/61.5=53.8). Further extrapolating from Table 2.0 shows that resilient member surface area b occupies from about 46.2% to about 56.6% of the body region surface area (28.4/61.5=46.2 and 32.6/57.6=56.6).

TABLE 2.0

| | engagement | engagement | engagement | resilient | body |
|---|---|---|---|---|---|
| | element | element | element | member | region |
| % of total plan view | surface area | surface area | surface area | surface area | surface |
| surface area | (a + d) | (a + c + d) | (c) | (b) | area |
| prior art 3-band | 40.1% | 47.1% | 7.0% | 52.9% | 59.9% |
| dilator 10 | 42.4% | 67.4% | 25.0% | 32.6% | 57.6% |
| FIGS. 1-11 | | (1.43 × more) | (3.57 × more) | (1.62 × less) | |
| prior art 2-band | 39.4% | 51.4% | 12.0% | 48.6% | 60.6% |
| dilator 10 | 38.5% | 71.6% | 33.1% | 28.4% | 61.5% |
| FIGS. 12-17 | | (1.39 × more) | (2.76 × more) | (1.71 × less) | |
| average for | 40.1% | | | | 59.9% |
| all dilators | | | | | |

Table 2.1 summarizes the ratios between combined engagement element surface areas a, c and d and resilient member surface areas b.

TABLE 2.1

| | engagement | resilient | | |
|---|---|---|---|---|
| | element | member | ratio | |
| % of total plan view | surface area | surface area | (a + c + d) | times |
| surface area | (a + c + d) | (b) | to (b) | greater |
| prior art 3-band | 47.1% | 52.9% | 0.887:1 | |
| dilator 10 FIGS. 1-11 | 67.4% | 32.6% | 2.068:1 | 2.33 |
| prior art 2-band | 51.4% | 48.6% | 1.058:1 | |
| dilator 10 FIGS. 12-17 | 71.6% | 28.4% | 2.521:1 | 2.38 |

Table 2.2 summarizes the above-described greater permeable engagement element surface area of dilator compared to the prior art nasal dilators:

TABLE 2.2

| | engagement | | engagement | | |
|---|---|---|---|---|---|
| | element | surface | element | surface | distance s |
| % of total plan view | surface area | area times | surface area | area times | (width) times |
| surface area | (a + c + d) | greater | (c) | greater | greater |
| prior art 3-band | 47.1% | | 7.0% | | |
| dilator 10 | 67.4% | 1.43 | 25.0% | 3.57 | 3.6 |
| FIGS. 1-11 | | | | | |
| prior art 2-band | 51.4% | | 12.0% | | |
| dilator 10 | 71.6% | 1.39 | 33.1% | 2.76 | 2.7 |
| FIGS. 12-17 | | | | | |

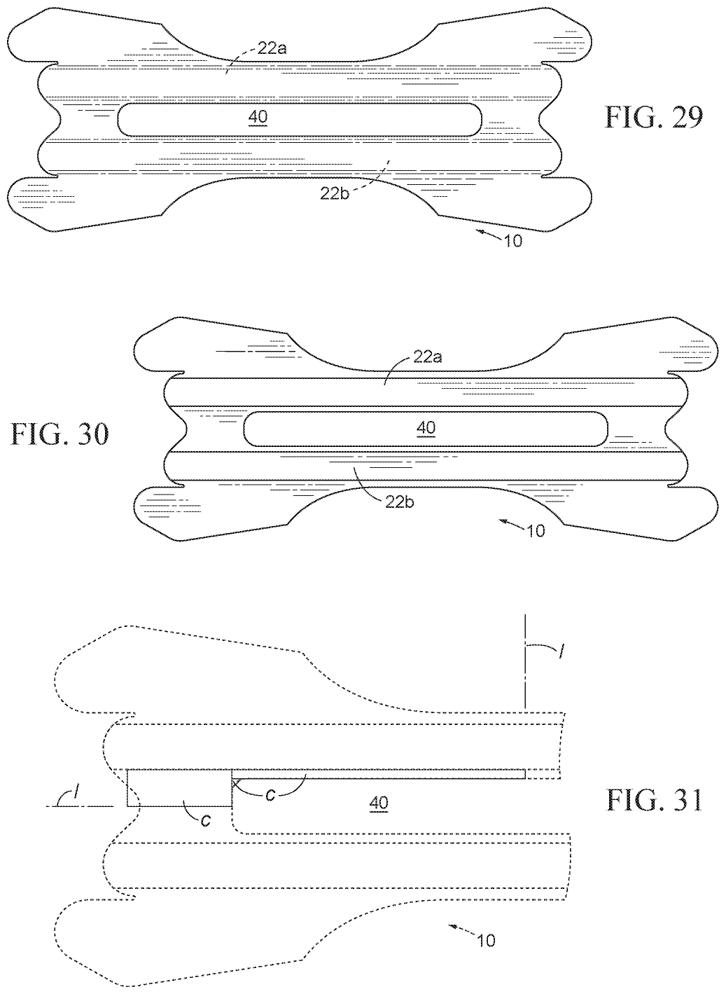

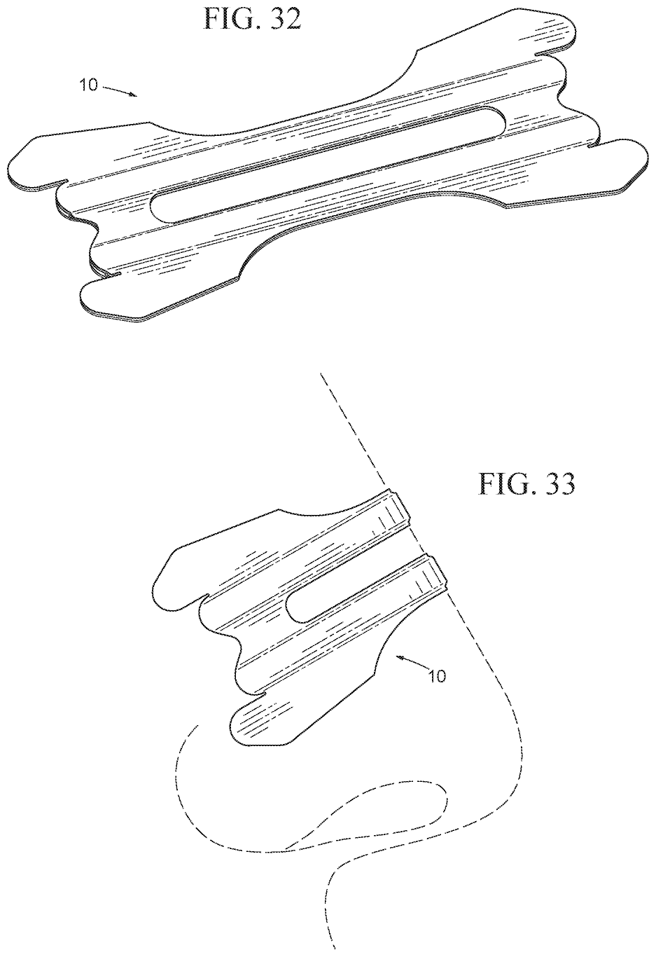

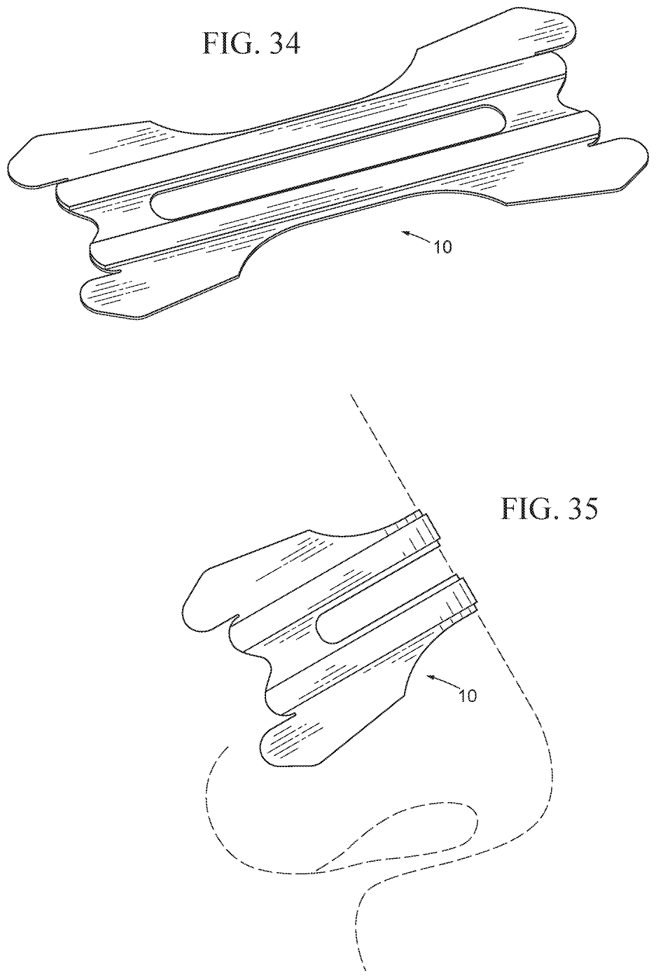

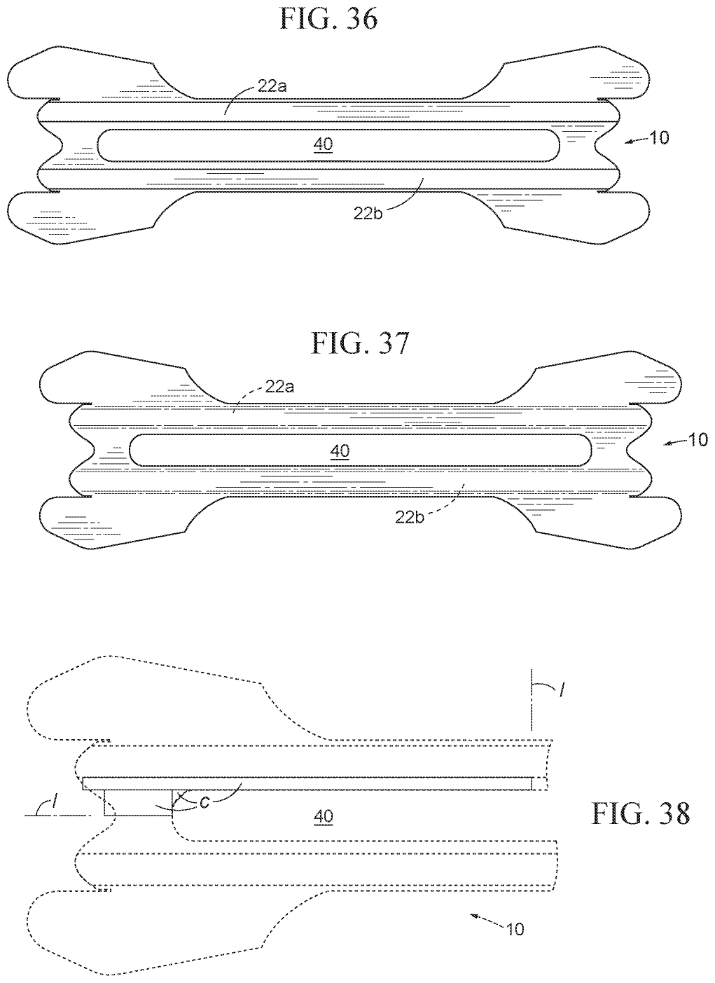

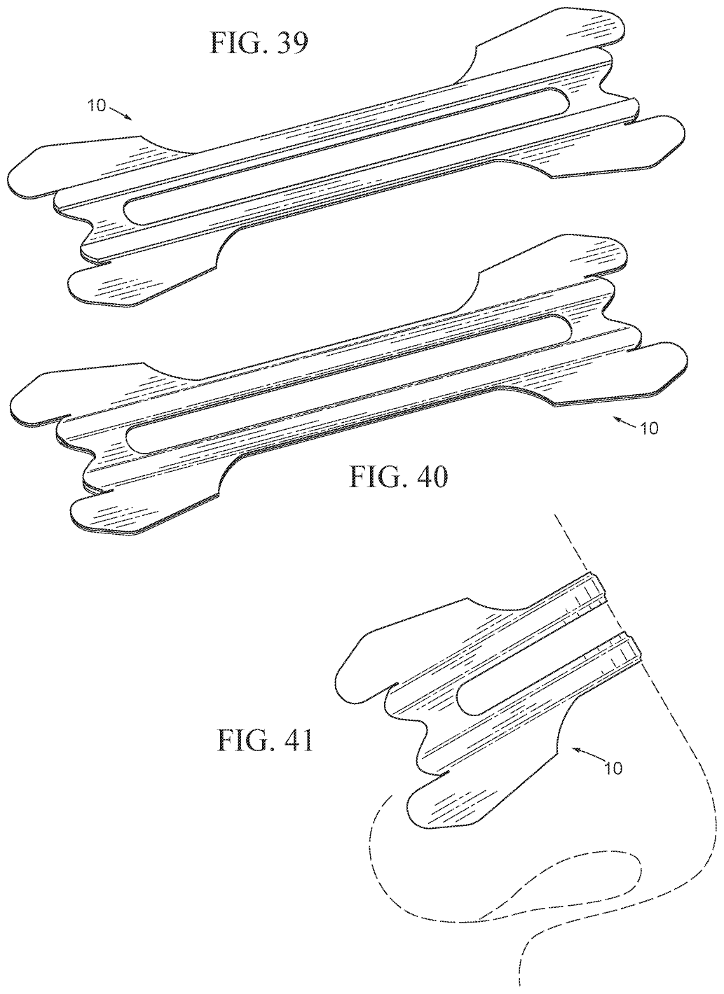

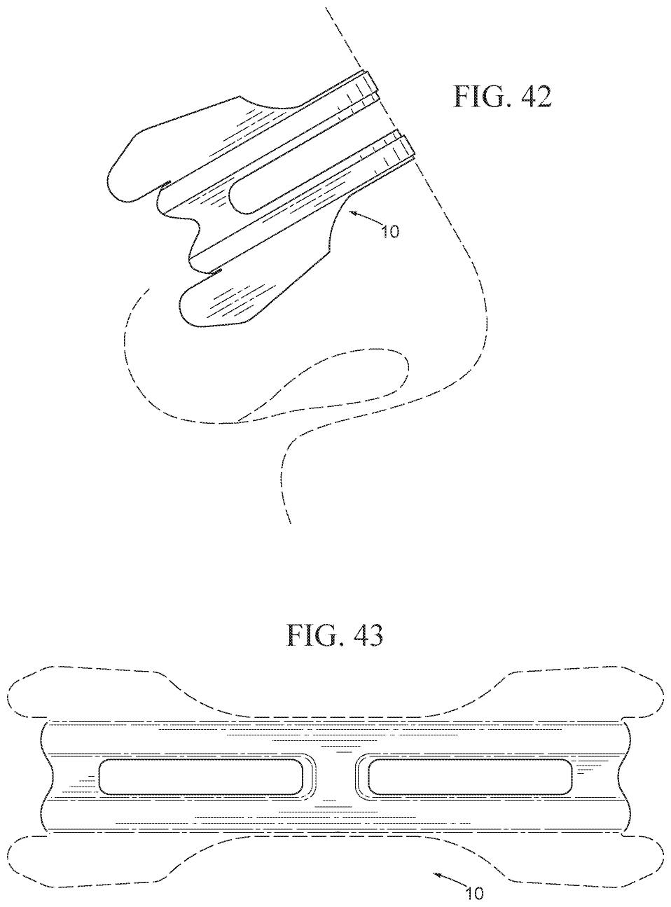

To further increase breathability, FIGS. 29-42 illustrate that dilator 10 may include an opening, 40, within engagement element surface area c. As particularly seen in FIGS. 31 and 38, opening 40 may displace roughly one-half of surface area c, as seen in Table 2.3.

TABLE 2.3

| | | | resilient member |
|---|---|---|---|
| % of total plan view | | surface areas | (impermeable) |
| surface area | opening 40 | (a + c + d) | surface areas (b) |
| prior art 3-band | n/a | 47.0% | 52.9% |
| dilator 10 | 12.2% | 55.2% | 32.6% |
| FIG. 25 | | | |
| prior art 2-band | n/a | 51.4% | 48.6% |
| dilator 10 | 15.2% | 56.4% | 28.4% |
| FIG. 28 | | | |

As a result of opening 40, impermeable resilient member surface areas b remain the same as before for both dilator 10 and the prior art nasal dilators. The combined engagement element surface areas a, c and d remain the same as before for the prior art nasal dilators (because they do not have an opening). However, where surface area c was previously 25.0% of the plan view surface area of dilator 10, FIG. 31 shows that opening 40 comprises 12.2% thereof, with the remainder of area c being 12.8% (12.8%+12.2%=25.0%). Similarly, FIG. 38 shows that opening 40 comprises 15.2% with the remainder of area c being 17.9% (17.9%+15.2%=33.1%).

In view of the increased breathable surface area of dilator 10, a hypothetical MVTR, both with and without opening 40, may be estimated and compared between the prior art nasal dilators and dilator 10 by assigning MVTR values to the previously discussed nasal dilator surface areas.

An MVTR value for the impermeable resilient member surface areas b is obviously zero. MVTR for engagement element surface areas a, c and d is based on a rate of 200 grams per square meter over 24 hours (GSM/24 hr.). The MVTR of human skin is believed to be ˜200-400 GSM/24 hr. (“What is MVTR and how is it measured?”, Avery Dennison Specialty Tape Division, U.S. Avery Dennison Corporation, 2018, stus.averydennison.com/std/stus.nsf/ed/). For purposes herein, MVTR for opening 40 is based on a rate of 400 GSM/24 hr.

A total plan view surface area of 11.5 square centimeters is used for the prior art 3-band nasal dilator, and 9.9 square centimeters for dilator 10 that corresponds to the prior art 2-band nasal dilator. These surface areas approximate nasal dilator actual size. As discussed hereinbefore, the prior art 2-band nasal dilator surface area was calculated to be about 0.5% less than corresponding dilator 10, and the surface area of dilator 10 corresponding to the prior art 3-band nasal dilator was calculated to be about 2.7% lesser.

Accordingly, surface area differences are reflected in the Total Surface Area column of Table 3.0. MVTR rates are represented as grams per square centimeter (Gcm\hat{ }2/24 hr.), which translates to 0.02 for engagement surface areas and 0.04 for opening 40. At 0.15 Gcm\hat{ }2/24 hr. dilator 10 has more than one-third greater MVTR (36.4%) than the prior art 3-band nasal dilator. At 0.14 Gcm\hat{ }2/24 hr. dilator 10 has about 40% greater MVTR than the prior art 2-band nasal dilator. Where dilator 10 includes opening 40, MVTR is roughly two-thirds greater; at 0.18 and 0.17 Gcm\hat{ }2/24 hr., MVTR is 63.6% and 70% greater, respectively.

Total surface areas, engagement surface area and MVTR values are summarized here:

TABLE 3.0

| | total | engagement | | | increase in | |
|---|---|---|---|---|---|---|
| | surface area | surface area | MVTR | greater | MVTR | greater |
| | a + b + c + d | a + c + d | Gcm\hat{ }2/24 hr | MVTR | w/opening | MVTR |
| | (cm\hat{ }2) | (cm\hat{ }2) | (a + c + d) | as % | 40 | as % |
| prior art 3-band | 11.5 | 5.41 | 0.11 | n/a | | |
| dilator 10 | 11.2 | 7.55 | 0.15 | 36.4% | 0.18 | 63.6% |
| | (2.7% less) | | | | | |
| prior art 2-band | 9.85 | 4.83 | 0.10 | n/a | | |
| | (0.5% less) | | | | | |
| dilator 10 | 9.9 | 7.08 | 0.14 | 40.0% | 0.17 | 70.0% |

It may be apparent to those of ordinary skill in the art that breathable materials used in medical devices may have an MVTR that is greater than 200 GSM/24 hr. However, the preferred PU film material for nasal dilators of the present invention has a thickness so as to be dimensionally stable, as described hereinbefore. In view of that preferred thickness it is believed that 200 GSM/24 hr. is a more realistic value. Similarly, fabric-based dilators typically utilize two nonwoven layers, each with an adhesive coating on one side. It is thus believed that 200 GSM/24 hr. is a reasonable estimated value for nonwoven dilator construction.

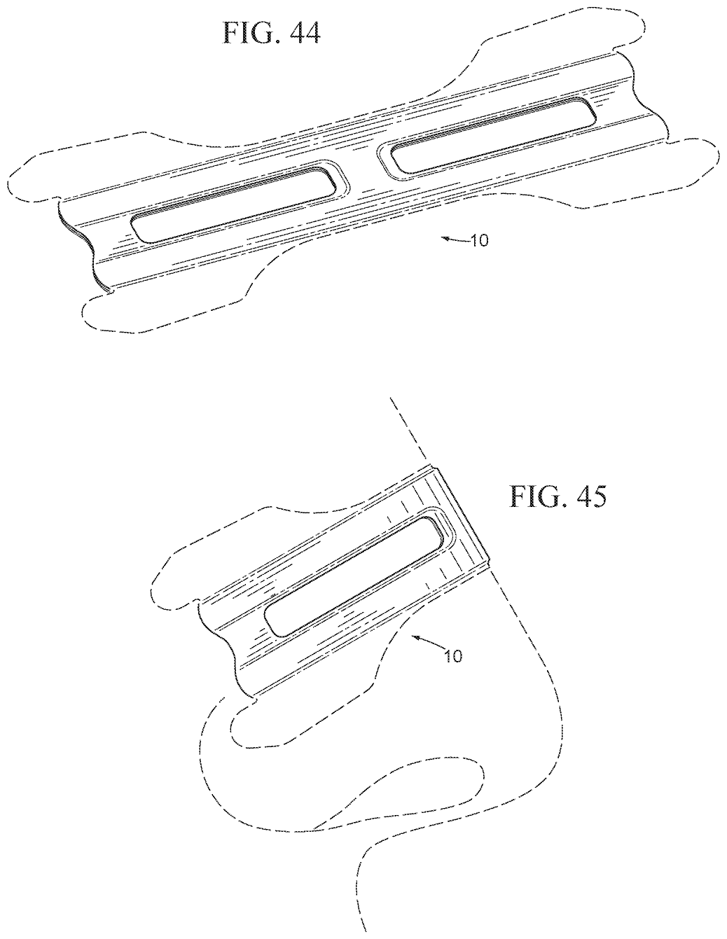

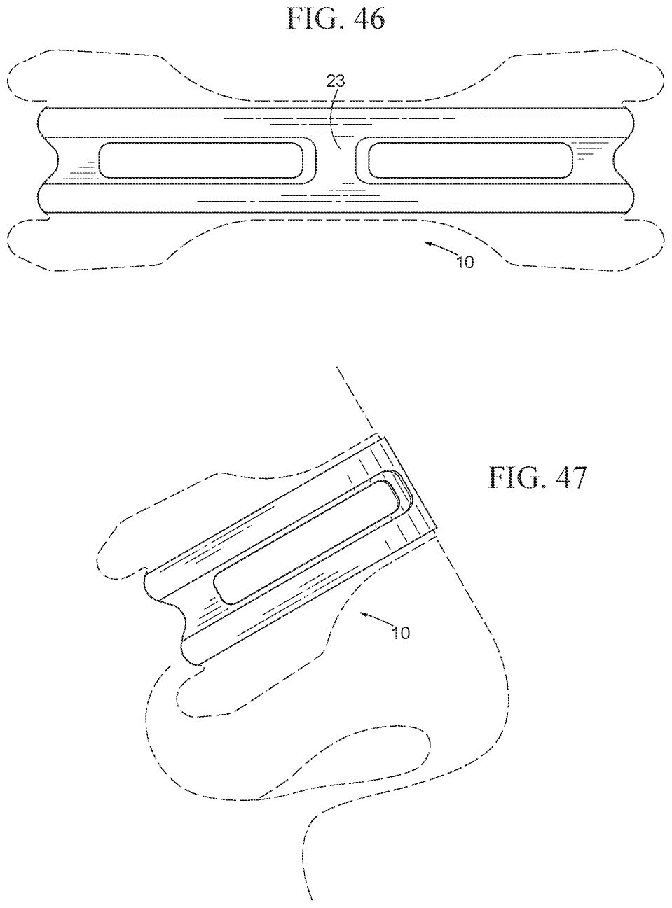

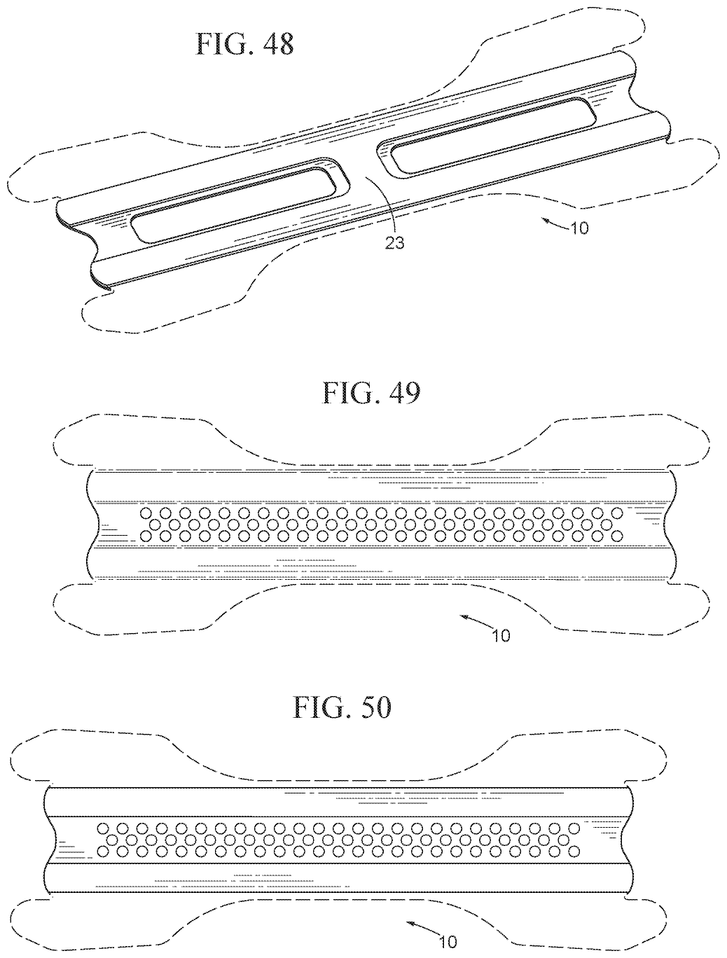

FIGS. 43-48 show that resilient members 22a and 22b may be joined together to form a single resilient member 22, preferably at mid-sections thereof, by a web, 23. Web 23 is preferably as narrow as practicable so as to displace as little of engagement element surface area c as possible. Opening 40 may be placed to either or both sides of web 23.

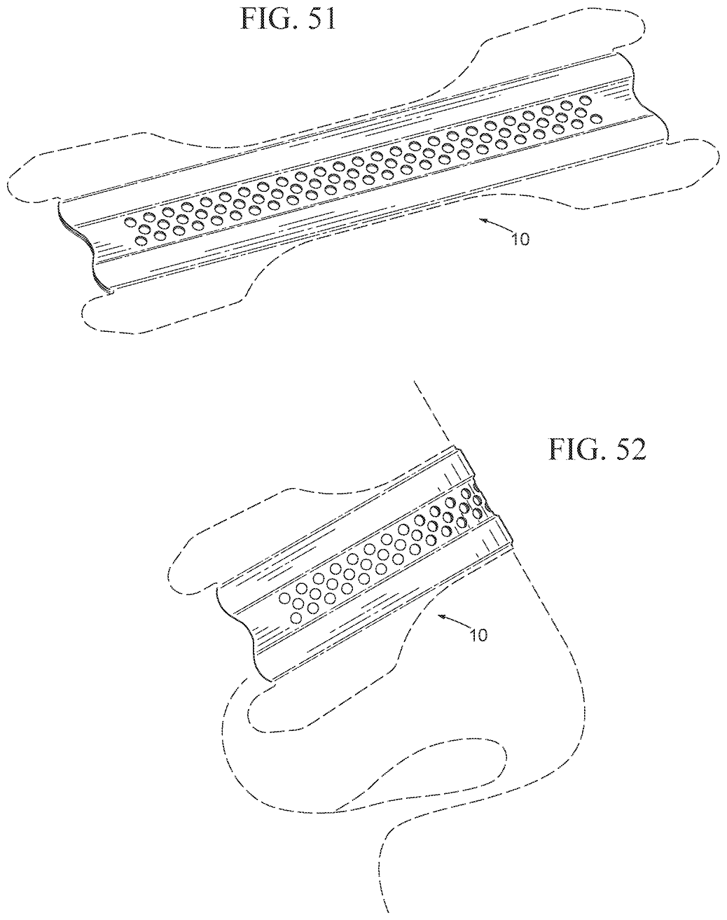

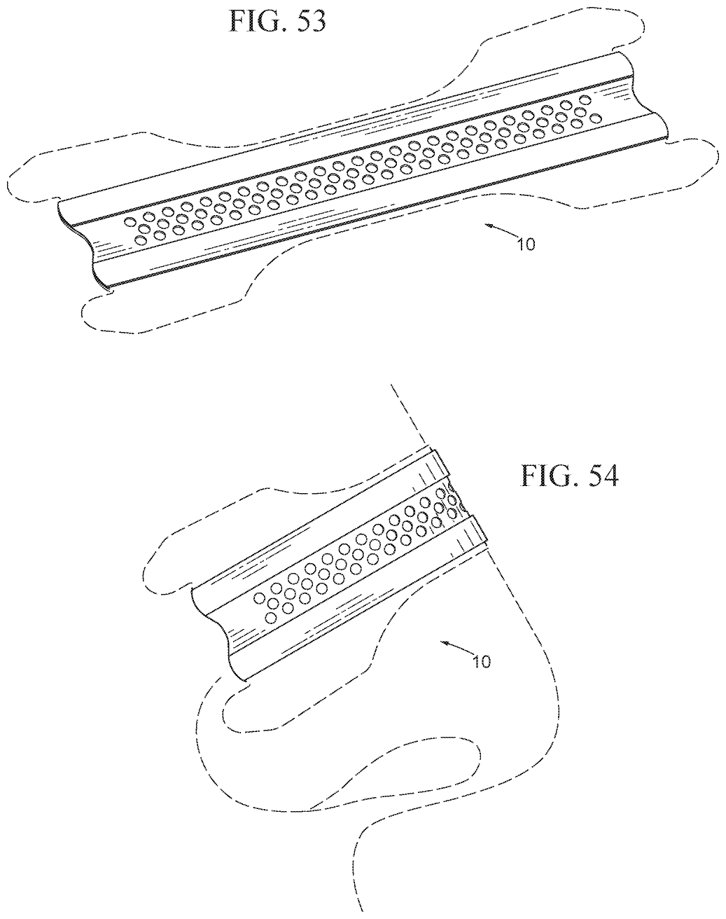

FIGS. 49-54 show that a plurality of small openings may be used in lieu of a one or two openings 40, or in lieu of microporous openings (perforations in thin supple films, such as PE film, as discussed hereinbefore). The size, shape and number of the smaller openings may vary from that shown in the drawing figures.

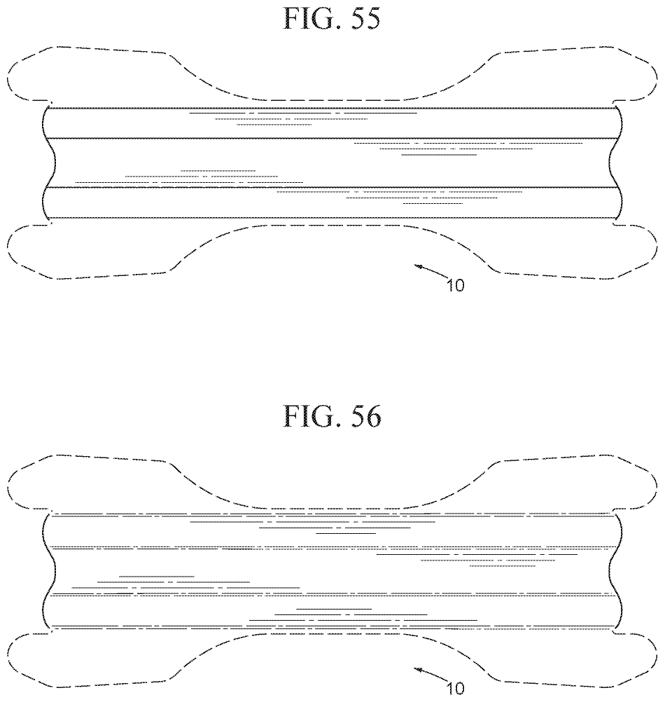

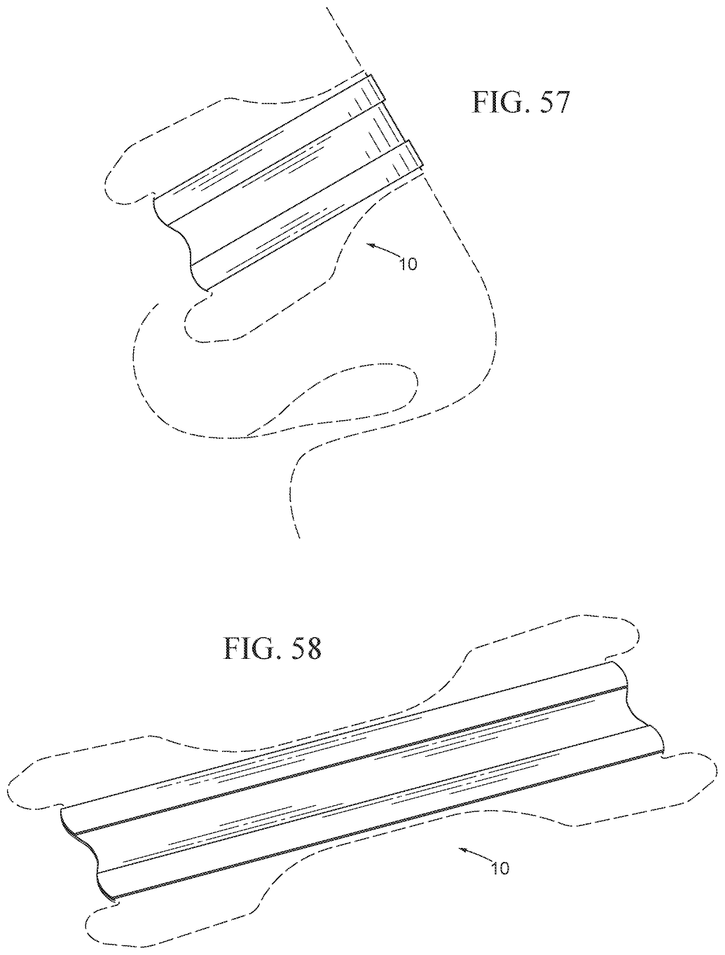

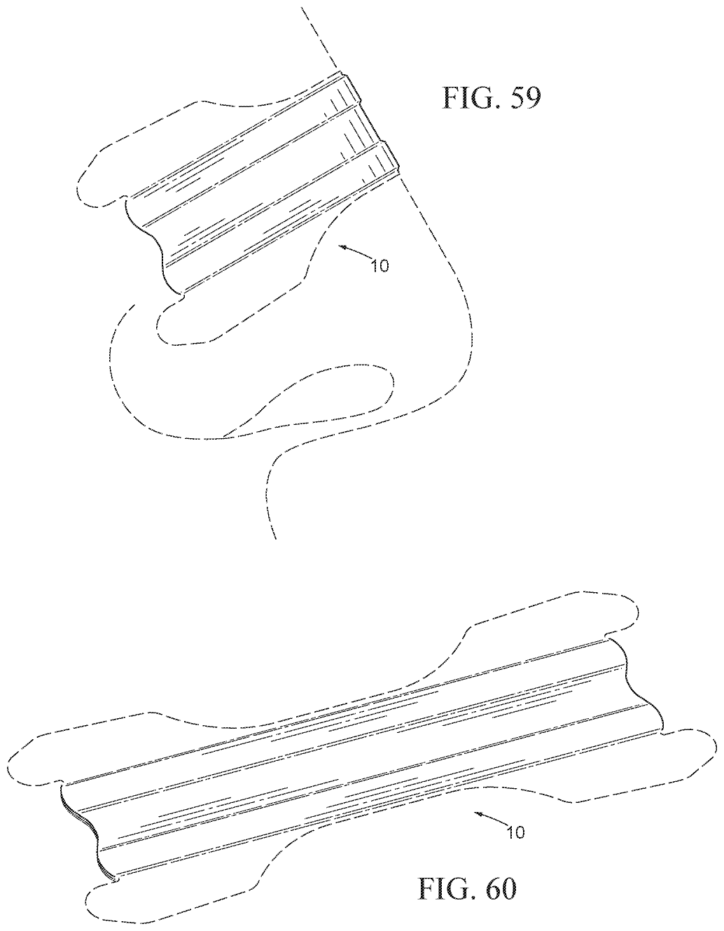

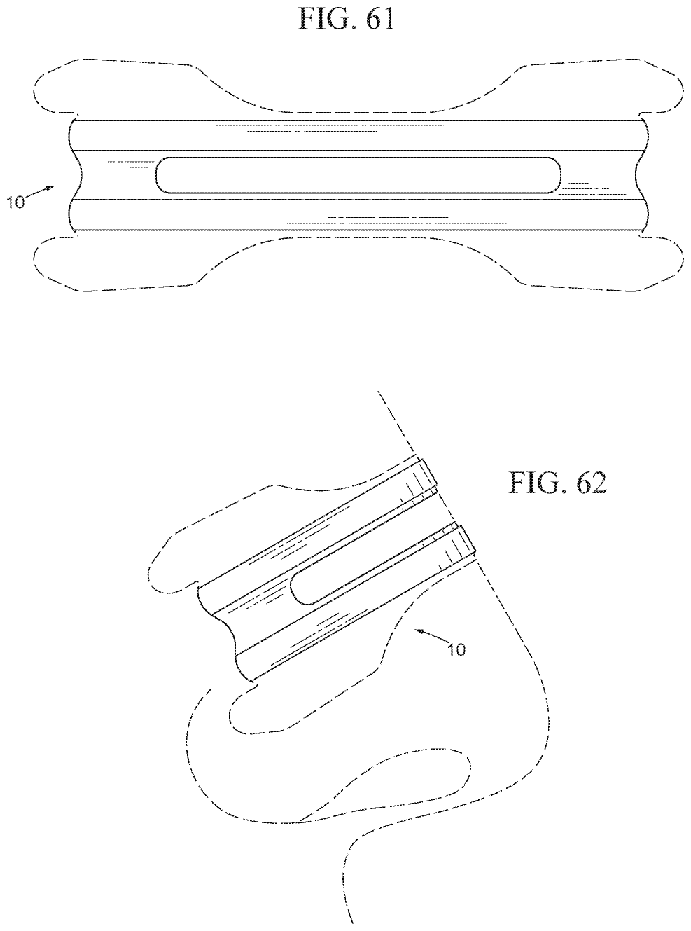

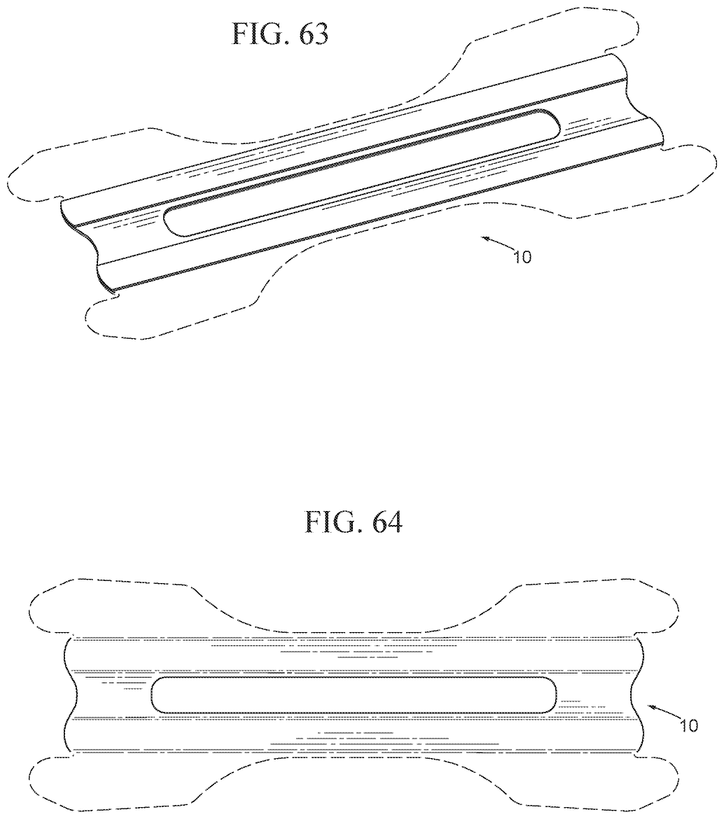

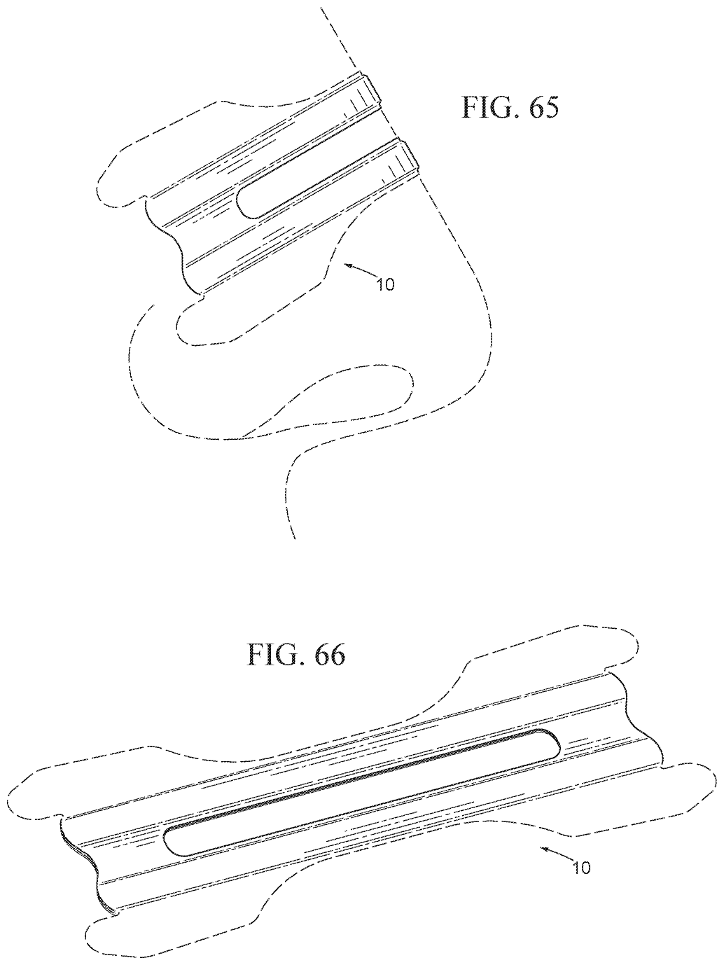

FIGS. 55-60 show an embodiment in accordance with the present invention.

1. A nasal dilator having a plan view periphery and a total width, length, and surface area, comprising:
a central oblong body region extending the total length of the nasal dilator, the body region having a width not greater than 45% of the dilator width and a body region surface area;
an impermeable surface area contained entirely within the body region; and
a permeable surface area;
wherein a portion of the permeable surface area is contained within the body region, said portion being from about 43% to about 54% of the body region surface area; and
wherein a portion of the permeable surface area is contained outside the body region, said portion being from about 38% to about 43% of the nasal dilator surface area.

2. The nasal dilator of claim 1, further comprising at least one resilient member having a periphery and a width, said periphery defining the impermeable surface area.

3. The nasal dilator of claim 2, wherein the width of the at least one resilient member is from about 45% to about 55% of the body region width; and
wherein the surface area of the at least one resilient member is from about 46% to about 57% of the body region surface area.

4. The nasal dilator of claim 2, wherein the width of each of the at least one resilient member is less than 0.125″ and a thickness thereof is greater than 0.010″.

5. The nasal dilator of claim 2, wherein the at least one resilient member consists of two or three resilient members spaced laterally apart; and
wherein a combined width of the two or three resilient members is from about one-fifth to about one-fourth of the nasal dilator width.

6. The nasal dilator of claim 2, wherein the at least one resilient member consists of two resilient members; and
wherein a combined width of the two resilient members is about 19% of the nasal dilator width.

7. The nasal dilator of claim 2, wherein the at least one resilient member consists of two resilient members laterally spaced apart by a distance greater than a combined width of the two resilient members; and
wherein the body region width is about 2.2 times greater than the combined width of the two resilient members.

8. The nasal dilator of claim 2, further comprising:
a first corner tab region abutting a first long edge of the body region; and
a second corner tab region abutting a second long edge of the body region,
the first and second corner tab regions having a surface area and extending the full length of the nasal dilator; and
wherein a total surface area of the first and second corner tab regions combined is about 40% of the nasal dilator surface area, the surface area of the body region is about 60% of the nasal dilator surface area.

9. The nasal dilator of claim 8, wherein the impermeable surface area is from about 1.6 to about 1.7 times lesser and the permeable surface area is from about 1.35 to about 1.45 times greater compared to a similar nasal dilator having a substantially same periphery and surface area, two or three resilient members positioned wholly within a same-sized body region having a surface area of about 60% of nasal dilator surface area, and a corner tab region surface area of about 40% of the nasal dilator surface area.

10. The nasal dilator of claim 9, wherein a ratio between the permeable surface area and the impermeable surface area is from about 2:1 to about 2.5:1; and
wherein said ratio is from about 2.3 to 2.4 times greater than the similar nasal dilator, such that the moisture vapor transmission rate of the nasal dilator is from about 36% to about 40% greater than the similar nasal dilator.

11. The nasal dilator of claim 9, wherein the permeable body region surface area is from about 2.7 to about 3.6 times greater than the similar nasal dilator, such that a moisture vapor transmission rate of the nasal dilator is from about 36% to about 40% greater than the similar nasal dilator.

12. The nasal dilator of claim 11, further comprising:
at least one opening extending vertically through the body region surface area, the opening positioned adjacent the at least one resilient member,
wherein the opening displaces a portion of the body region permeable surface area, such that the moisture vapor transmission rate of the nasal dilator is roughly 66% greater than the similar nasal dilator.

13. The nasal dilator of claim 1, wherein the engagement element comprises at least one layer selected from the group consisting of:
i) a base member, wherein the base member is a peripheral defining layer of the nasal dilator;
ii) a cover member, wherein the cover member is the peripheral defining layer of the nasal dilator; and
iii) at least one base member and a cover member, wherein the cover member or the at least one base member is the peripheral defining layer of the nasal dilator.
